SEQUENCE LISTING (85 sequences)

SEQ ID NO: 1 - protein, 124 aa, Artificial Sequence
  heavy chain variable domain 1
QVQLLQSGAEVKKPGASVKVSCKASGYTFTSYGISWVRQAPGQGLEWVGWINTYSGGTKYAQKFQGRVTMTRDTSISTVYMELSGLKSDDTAVYYCARLGHCQRGICSDALDTWGQGTLVTVSS

SEQ ID NO: 2 - protein, 124 aa, Artificial Sequence
  heavy chain variable domain 2
EVQLLQSGAEVKEPGASVKVSCKASGYTFSSYGISWVRQAPGQGLEWVGRINTYNGNTGYAQRLQGRVTMTTDTSTSIAYMEVRSLRSDDTAVYYCARLGHCQRGICSDALDTWGQGTMVTVSS

SEQ ID NO: 3 - protein, 125 aa, Artificial Sequence
  heavy chain variable domain 3
QVQLVQSGGGVVQTGGSLRLSCAASGFTFRNYGIHWVRQAPGKGLEWVASISYDGSNKYYADSVKGRFTISRDNSRNTVHVQMDSLRGGDTAVYYCARDVHYYGSGSYYNAFDIWGQGTLVTVSS

SEQ ID NO: 4 - protein, 120 aa, Artificial Sequence
  heavy chain variable domain 4
QVQLVQSGGGLVQPGGSLRLSCAASGFTFSSYAMSWVRQAPGKGLEWLSVISHDGGFQYYADSVKGRFTVSRDNSKNTLYLQMNSLRAEDTAVYYCARAGWLRQYGMDVWGQGTLVTVSS

SEQ ID NO: 5 - protein, 117 aa, Artificial Sequence
  heavy chain variable domain 5
EVQLVQSGTEVKKPGESLKISCKISGYSFTAYWIAWVRQMPGKGLEWMGMIWPPDADARYSPSFQGQVTFSVDKSISTAYLQWHSLKTSDTAVYYCARLYSGSYSPWGQGTLVTVSS

SEQ ID NO: 6 - protein, 120 aa, Artificial Sequence
  heavy chain variable domain 6
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSS

SEQ ID NO: 7 - protein, 108 aa, Artificial Sequence
  light chain variable domain 1
ELVLTQSPSSLSASVGDRVTITCRASQSISRYLNWYQQKPGKAPKLLIYGASRLESGVPSRFSGSGSGTDFTLTISSLQPEDFATYYCQQSDSVPVTFGQGTRLEIKR

SEQ ID NO: 8 - protein, 109 aa, Artificial Sequence
  light chain variable domain 2
DIVLTQSPSSLSASVGDRVTITCRASQSISSYLNWYQQKPGKAPKLLIYAASSLQSGVPSRFSGSGSGTDFTLTISSLQPEDFATYYCQQSYSTPPYTFGQGTKLEIKR

SEQ ID NO: 9 - protein, 110 aa, Artificial Sequence
  light chain variable domain 3
ELVLTQSPGTLSLSPGERATLSCRASQSIFNYVAWYQQKPGQAPRLLIYDASNRATGIPARFSGSGSGTDFTLTISSLEPEDFAVYYCQQRSKWPPTWTFGQGTRVDIKR

SEQ ID NO: 10 - protein, 109 aa, Artificial Sequence
  light chain variable domain 4
ELVLTQSPGTLSLSPGERATLSCRASETVSSRQLAWYQQKPGQAPRLLIYGASSRATGIPDRFSGSGSGTDFTLTISRLEPEDSAVFYCQQYGSSPRTFGGGTKLEIKR

SEQ ID NO: 11 - protein, 109 aa, Artificial Sequence
  light chain variable domain 5
ELVLTQSPGTLSLSPGERATLSCRASQSVSSSSLAWYQQKPGQAPRLLIYGASSRATGIPDRFSGSGSGTDFTLTISSLQPEDAATYYCQKYSSYPLTFGQGTKLEIKR

SEQ ID NO: 12 - protein, 109 aa, Artificial Sequence
  light chain variable domain 6
ELVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 13 - protein, 109 aa, Artificial Sequence
  light chain variable domain 7
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKR

SEQ ID NO: 14 - protein, 102 aa, Artificial Sequence
  Feline heavy chain constant domain
ASTTAPSVFPLAPSSGTTSGATVALACLVLGYFPEPVTVSWNSGALTSGVHTFPAVLQASGLYSLSSMVTVPSSRWLSDTFTCNVAHPPSNTKVDKTVDRTD

SEQ ID NO: 15 - protein, 109 aa, Artificial Sequence
  Feline light chain constant domain
SDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSESQRE

SEQ ID NO: 16 - protein, 222 aa, Artificial Sequence
  FL355 heavy chain
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTTAPSVFPLAPSSGTTSGATVALACLVLGYFPEPVTVSWNSGALTSGVHTFPAVLQASGLYSLSSMVTVPSSRWLSDTFTCNVAHPPSNTKVDKTVDRTD

SEQ ID NO: 17 - protein, 218 aa, Artificial Sequence
  FL355 light chain
DIVLTQSPGTLSLSPGETATLSCRASQSVGSNLAWYQQKPGQAPRLLIYGASTGATGVPARFSGSRSGTDFTLTITSLQPEDFATYYCQQYYSFLAKTFGQGTQLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSESQRE

SEQ ID NO: 18 - protein, 174 aa, Artificial Sequence
  fG-CSF
TPLGPTSSLPQSFLLKCLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPTQGTMPTFTSAFQRRAGGTLVASNLQSFLEVAYRALRHFTKP

SEQ ID NO: 19 - protein, 175 aa, Artificial Sequence
  mutant fG-CSF
ATPLGPTSSLPQSFLLKSLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPAQGTMPTFTSAFQRRAGGTLVASNLQSFLEVAYRALRHFTKP

SEQ ID NO: 20 - protein, 15 aa, Artificial Sequence
  linker 1
GSAGSAPAPAGSGEF

SEQ ID NO: 21 - protein, 10 aa, Artificial Sequence
  linker 2
GGGGSGGGGS

SEQ ID NO: 22 - protein, 411 aa, Artificial Sequence
  ABP-F1
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTTAPSVLPLAPSSGTTSGATVALACLVLGYFPEPVTVSWNSGALTSGVHTFPAVLQASGLYSLSSMVTVPSSRWLSDTFTCNVAHPPSNTKVDKTVDRTDGSAGSAPAPAGSGEFTPLGPTSSLPQSFLLKCLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPTQGTMPTFTSAFQRRAGGTLVASNLQSFLEVAYRALRHFTKP

SEQ ID NO: 23 - protein, 407 aa, Artificial Sequence
  ABP-F1_2
QVQLVQSGGGPVKPGGSLRLSCAASGFMFRAYSMNWVRQAPGKGLEWVSSISSSGRYIHYADSVKGRFTISRDNAKNSLYLQMNSLRAEDTAVYYCARETVMAGKALDYWGQGTLVTVSSASTTAPSVFPLAPSSGTTSGATVALACLVLGYFPEPVTVSWNSGALTSGVHTFPAVLQASGLYSLSSMVTVPSSRWLSDTFTCNVAHPPSNTKVDKTVDRTDGGGGSGGGGSATPLGPTSSLPQSFLLKSLEQVRKVQADGTALQERLCAAHKLCHPEELVLLGHALGIPQAPLSSCSSQALQLTGCLRQLHSGLFLYQGLLQALAGISPELAPTLDMLQLDITDFAINIWQQMEDVGMAPAVPPAQGTMPTFTSAFQRRAGGTLVASNLQSFLEVAYRALRHFTKP

SEQ ID NO: 24 - protein, 540 aa, Artificial Sequence
  Feline serum albumin
RGVTRREAHQSEIAHRFNDLGEEHFRGLVLVAFSQYLQQCPFEDHVKLVNEVTEFAKGCVADQSAANCEKSLHELFGDKLCTVASLRDKYGEMADCCEKKEPERNECFLQHKDDNPGFGQLVTPEADAMCTAFHENEQRFLGKYLYEIARRHPYFYAPELLYYAEEYKGVFTECCEAADKAACLTPKVDALREKVLASSAKERLKCASLQKFGERAFKAWSVARLSQKFPKAEFAEISKLVTDLAKIHKECCHGDLLECADDRADLAKYICENQDSISTKLKECCGKPVLEKSHCISEVERDELPADLPPLAADFVEDKEVCKNYQEAKDVFLGTFLYEYSRRHPEYSVSLLLRLAKEYEATLEKCCATDDPPACYAHVFDEFKPLVEEPHNLVKTNCELFEKLGEYGFQNALLVRYTKKVPQVSTPTLVEVSRSLGKVGSKCCTHPEAERLSCAEDYLSVVLNRLCVLHEKTPVSERVTKCCTESLVNRRPCFALQVDETYVPKEFSAETFTFHADLCTLPEAEKQIKKQSALVELLKH

SEQ ID NO: 25 - DNA, 37 bp, Artificial Sequence
  primer 1
gatcaactctagagccaccatggagtggtcctgggtc

SEQ ID NO: 26 - DNA, 22 bp, Artificial Sequence
  primer 2
tgagctgactgtgaccagagtg

SEQ ID NO: 27 - DNA, 36 bp, Artificial Sequence
  primer 3
ggtcacagtcagctcagcttctaccaccgcaccttc

SEQ ID NO: 28 - DNA, 56 bp, Artificial Sequence
  primer 4
aggaagacgcttttagaggcggccgctcagtcggttcggtccactgttttatcgac

SEQ ID NO: 29 - DNA, 25 bp, Artificial Sequence
  primer 5
gacatcgtcctgacccagagccccg

SEQ ID NO: 30 - DNA, 24 bp, Artificial Sequence
  primer 6
ccgcttgatctccagctgggtgcc

SEQ ID NO: 31 - DNA, 37 bp, Artificial Sequence
  primer 7
cagctggagatcaagcggagtgacgcacaaccttctg

SEQ ID NO: 32 - DNA, 48 bp, Artificial Sequence
  primer 8
aggaagacgcttttagagctactccctctgggactcgctccgattaaa

SEQ ID NO: 33 - DNA, 54 bp, Artificial Sequence
  primer 9
accaccggagtgctttccacccccctgggaccaacctccagcctgccccagtcc

SEQ ID NO: 34 - DNA, 60 bp, Artificial Sequence
  primer 10
atcggcggccgcgaagacgcttttagatcagtggtggtggtgatggtggtggtgcccagg

SEQ ID NO: 35 - DNA, 49 bp, Artificial Sequence
  primer 11
gcgtgaccaccggagtgctttccgcaaccccactgggaccaaccagttc

SEQ ID NO: 36 - DNA, 84 bp, Artificial Sequence
  primer 12
atcggcggccgcgaagacgcttttagatcagtggtggtggtgatggtggtggtgccctggcttggtaaagtggcgaagagcccg

SEQ ID NO: 37 - DNA, 25 bp, Artificial Sequence
  primer 13
cttggagccagaggcaagacagaag

SEQ ID NO: 38 - DNA, 25 bp, Artificial Sequence
  primer 14
cttctgtcttgcctctggctccaag

SEQ ID NO: 39 - DNA, 32 bp, Artificial Sequence
  primer 15
aggaagacgcttttagaggcggccgctcaagg

SEQ ID NO: 40 - protein, 5 aa, Artificial Sequence
  linker
GGGGS

SEQ ID NO: 41 - protein, 5 aa, Artificial Sequence
  linker
SGGGG

SEQ ID NO: 42 - protein, 5 aa, Artificial Sequence
  linker
SRSSG

SEQ ID NO: 43 - protein, 5 aa, Artificial Sequence
  linker
SGSSC

SEQ ID NO: 44 - protein, 12 aa, Artificial Sequence
  linker
GKSSGSGSESKS

SEQ ID NO: 45 - protein, 6 aa, Artificial Sequence
  linker
RPPPPC

SEQ ID NO: 46 - protein, 7 aa, Artificial Sequence
  linker
SSPPPPC

SEQ ID NO: 47 - protein, 14 aa, Artificial Sequence
  linker
GSTSGSGKSSEGKG

SEQ ID NO: 48 - protein, 18 aa, Artificial Sequence
  linker
GSTSGSGKSSEGSGSTKG

SEQ ID NO: 49 - protein, 18 aa, Artificial Sequence
  linker
GSTSGSGKPGSGEGSTKG

SEQ ID NO: 50 - protein, 14 aa, Artificial Sequence
  linker
EGKSSGSGSESKEF

SEQ ID NO: 51 - protein, 5 aa, Artificial Sequence
  heavy chain variable domain
SYGIS

SEQ ID NO: 52 - protein, 17 aa, Artificial Sequence
  heavy chain complementarity determining domain 2 (CDR)
WINTYSGGTKYAQKFQG

SEQ ID NO: 53 - protein, 15 aa, Artificial Sequence
  heavy chain complementarity determining domain 3 (CDR3)
LGHCQRGICSDALDT

SEQ ID NO: 54 - protein, 17 aa, Artificial Sequence
  heavy chain CDR2
RINTYNGNTGYAQRLQG

SEQ ID NO: 55 - protein, 5 aa, Artificial Sequence
  heavy chain CDR1
NYGIH

SEQ ID NO: 56 - protein, 17 aa, Artificial Sequence
  heavy chain CDR2
SISYDGSNKYYADSVKG

SEQ ID NO: 57 - protein, 16 aa, Artificial Sequence
  heavy chain CDR3
DVHYYGSGSYYNAFDI

SEQ ID NO: 58 - protein, 5 aa, Artificial Sequence
  heavy chain CDR1
SYAMS

SEQ ID NO: 59 - protein, 17 aa, Artificial Sequence
  heavy chain CDR2
VISHDGGFQYYADSVKG

SEQ ID NO: 60 - protein, 11 aa, Artificial Sequence
  heavy chain CDR3
AGWLRQYGMDV

SEQ ID NO: 61 - protein, 5 aa, Artificial Sequence
  heavy chain CDR1
AYWIA

SEQ ID NO: 62 - protein, 17 aa, Artificial Sequence
  heavy chain CDR2
MIWPPDADARYSPSFQG

SEQ ID NO: 63 - protein, 8 aa, Artificial Sequence
  heavy chain CDR3
LYSGSYSP

SEQ ID NO: 64 - protein, 5 aa, Artificial Sequence
  heavy chain CDR1
AYSMN

SEQ ID NO: 65 - protein, 17 aa, Artificial Sequence
  heavy chain CDR2
SISSSGRYIHYADSVKG

SEQ ID NO: 66 - protein, 11 aa, Artificial Sequence
  heavy chain CDR3
ETVMAGKALDY

SEQ ID NO: 67 - protein, 11 aa, Artificial Sequence
  light chain CDR1
RASQSISRYLN

SEQ ID NO: 68 - protein, 7 aa, Artificial Sequence
  light chain CDR2
GASRLES

SEQ ID NO: 69 - protein, 9 aa, Artificial Sequence
  light chain CDR3
QQSDSVPVT

SEQ ID NO: 70 - protein, 11 aa, Artificial Sequence
  light chain CDR1
RASQSISSYLN

SEQ ID NO: 71 - protein, 7 aa, Artificial Sequence
  light chain CDR2
AASSLQS

SEQ ID NO: 72 - protein, 10 aa, Artificial Sequence
  light chain CDR3
QQSYSTPPYT

SEQ ID NO: 73 - protein, 11 aa, Artificial Sequence
  light chain CDR1
RASQSIFNYVA

SEQ ID NO: 74 - protein, 7 aa, Artificial Sequence
  light chain CDR2
DASNRAT

SEQ ID NO: 75 - protein, 11 aa, Artificial Sequence
  light chain CDR3
QQRSKWPPTWT

SEQ ID NO: 76 - protein, 12 aa, Artificial Sequence
  light chain CDR1
RASETVSSRQLA

SEQ ID NO: 77 - protein, 7 aa, Artificial Sequence
  light chain CDR2
GASSRAT

SEQ ID NO: 78 - protein, 9 aa, Artificial Sequence
  light chain CDR3
QQYGSSPRT

SEQ ID NO: 79 - protein, 12 aa, Artificial Sequence
  light chain CDR1
RASQSVSSSSLA

SEQ ID NO: 80 - protein, 9 aa, Artificial Sequence
  light chain CDR3
QKYSSYPLT

SEQ ID NO: 81 - protein, 11 aa, Artificial Sequence
  light chain CDR1
RASQSVGSNLA

SEQ ID NO: 82 - protein, 7 aa, Artificial Sequence
  light chain CDR2
GASTGAT

SEQ ID NO: 83 - protein, 10 aa, Artificial Sequence
  light chain CDR3
QQYYSFLAKT

SEQ ID NO: 84 - protein, 15 aa, Artificial Sequence
  Linker aa
GSAGSAPAPAGSGEF

SEQ ID NO: 85 - protein, 10 aa, Artificial Sequence
  Linker aa
GGGGSGGGGS

(19) United States
(12) Patent Application Publication
(10) Pub. No.: US 2023/0140224 A1
(43) Pub. Date: May 4, 2023

(54) RECOMBINANT PROTEINS COMPRISING FELINE GRANULOCYTE COLONY-STIMULATING FACTOR AND ANTIGEN BINDING FRAGMENT FOR SERUM ALBUMIN, AND USES THEREOF

(71) Applicant: APRILBIO CO., LTD., Chuncheon-si, Gangwon-do (KR)

(72) Inventor: Sang Hoon CHA, Chuncheon-si, Gangwon-do (KR)

(21) Appl. No.: 17/917,490

(22) PCT Filed: Apr. 9, 2021
(86) PCT No.: PCT/IB2021/052969

§ 371 Date: Oct. 6, 2022

(30) Foreign Application Priority Data
Apr. 9, 2020 (KR) 10-2020-0043606

(51) Int. Cl. C07K 16/18; C12N 15/85; C07K 14/535

(52) U.S. Cl. CPC C07K 16/18; C12N 15/85; C07K 14/535; C12N 2800/107; C07K 2317/565; C07K 2317/24; C07K 2319/31; C07K 2317/55; A61K 39/00

(57) ABSTRACT

The present disclosure relates to recombinant proteins comprising a feline granulocyte colony-stimulating factor and an antigen binding fragment that binds to serum albumin, nucleic acid molecules encoding the recombinant proteins, vectors, cells, and uses thereof. Provided are compositions for treating feline panleukopenia.

### CROSS-REFERENCE TO RELATED APPLICATIONS

This application claims priority to KR Appl. No. 10-2020-0043606, filed Apr. 9, 2020, the disclosure of which is incorporated herein by reference in its entirety.

### REFERENCE TO SEQUENCE LISTING SUBMITTED ELECTRONICALLY

The content of the electronically submitted sequence listing in ASCII text file (Name: 2662-0002WO01_Sequence_Listing_ST25.txt; Size: 48 KB; and Date of Creation: Apr. 9, 2021) filed with the application is incorporated herein by reference in its entirety.

### FIELD

The present disclosure relates to recombinant proteins comprising a feline granulocyte colony-stimulating factor and an antigen binding fragment that binds to serum albumin, nucleic acid molecules encoding the recombinant proteins, vectors, cells, compositions, and uses thereof.

### BACKGROUND

Feline panleukopenia is a viral enteritis caused by feline parvovirus (FPV), which is highly contagious, has a high mortality rate, and is one of the most fatal diseases for all cat species. Current treatment of feline panleukopenia includes whole blood transfusion or granulocyte colony-stimulating factor (GCSF) administration to increase the number of white blood cells or intravenous administration of a fluid containing antibiotics and vitamins A, B, C, etc. to prevent dehydration due to sepsis. However, intravenous administration of a fluid containing antibiotics, etc. is not a direct treatment for the disease, and transfusion also has a problem in that it is difficult to secure a sufficient amount of whole blood. Although the administration of GCSF is widely used in the treatment of feline panleukopenia, recombinant GCSF used for treatment is hGCSF derived from a human, not a cat. Therefore, when used for a long period of time, anti-drug antibodies (ADAs) against hGCSF are produced, which not only reduces its medicinal effect or therapeutic efficacy, but also causes serious side effects such as acute immune responses, autoimmune diseases, etc.

In addition, feline GCSF, which is one of the hormones secreted in a cat's body, is known as a protein that regulates production of circulating blood cells in the bone marrow. Specifically, the GCSF stimulates proliferation and differentiation of neutrophils to increase neutrophil levels in the blood, thereby contributing to shortening the neutropenic period and to restoring immunity. However, this protein has an in vivo half-life of only about 4 hrs to about 5 hrs, and thus, multiple administrations are required to maintain therapeutic efficacy for a long time. For this reason, PEGylation, which conjugates a polymer such as a poly(alkylene glycol) derivative to GCSF, or hyperglycosylation is widely used (KR Patent Pub. No. 10-2010-0052501). This also entails problems, such as protein denaturation caused during a chemical fusion process and immunogenic potential.

### SUMMARY

Disclosed herein are recombinant proteins comprising (a) an antigen binding fragment comprising a heavy chain and a light chain and (b) a feline granulocyte colony-stimulating factor (fGCSF),

wherein the heavy chain comprises a heavy chain variable domain and a feline heavy chain constant 1 domain, wherein the heavy chain variable domain comprises

(1) a heavy chain complementarity determining domain 1 (CDR1) comprising the amino acid sequence of SYGIS (SEQ ID NO:51),

a heavy chain complementarity determining domain 2 (CDR) comprising the amino acid sequence of WINTYSGGTKYAQKFQG (SEQ ID NO:52), and

a heavy chain complementarity determining domain 3 (CDR3) comprising the amino acid sequence of LGHCQRGICSDALDT (SEQ ID NO:53);

(2) a heavy chain CDR1 comprising the amino acid sequence of SYGIS (SEQ ID NO:51),

a heavy chain CDR2 comprising the amino acid sequence of RINTYNGNTGYAQRLQG (SEQ ID NO:54), and

a heavy chain CDR3 comprising the amino acid sequence of LGHCQRGICSDALDT (SEQ ID NO:53);

(3) a heavy chain CDR1 comprising the amino acid sequence of NYGIH (SEQ ID NO:55),

a heavy chain CDR2 comprising the amino acid sequence of SISYDGSNKYYADSVKG (SEQ ID NO:56), and

a heavy chain CDR3 comprising the amino acid sequence of DVHYYGSGSYYNAFDI (SEQ ID NO:57),

(4) a heavy chain CDR1 comprising the amino acid sequence of SYAMS (SEQ ID NO:58),

a heavy chain CDR2 comprising the amino acid sequence of VISHDGGFQYYADSVKG (SEQ ID NO:59), and

a heavy chain CDR3 comprising the amino acid sequence of AGWLRQYGMDV (SEQ ID NO:60);

(5) a heavy chain CDR1 comprising the amino acid sequence of AYWIA (SEQ ID NO:61),

a heavy chain CDR2 comprising the amino acid sequence of MIWPPDADARYSPSFQG (SEQ ID NO:62), and

a heavy chain CDR3 comprising the amino acid sequence of LYSGSYSP (SEQ ID NO:63); or

(6) a heavy chain CDR1 comprising the amino acid sequence of AYSMN (SEQ ID NO:64),

a heavy chain CDR2 comprising the amino acid sequence of SISSSGRYIHYADSVKG (SEQ ID NO:65), and

a heavy chain CDR3 comprising the amino acid sequence of ETVMAGKALDY (SEQ ID NO:66); and

wherein the light chain comprises a light chain variable domain and a feline light chain constant domain, wherein the light chain variable domain comprises

(7) a light chain CDR1 comprising the amino acid sequence of RASQSISRYLN (SEQ ID NO:67),

a light chain CDR2 comprising the amino acid sequence of GASRLES (SEQ ID NO:68), and

a light chain CDR3 comprising the amino acid sequence of QQSDSVPVT (SEQ ID NO:69);

(8) a light chain CDR1 comprising the amino acid sequence of RASQSISSYLN (SEQ ID NO:70),

a light chain CDR2 comprising the amino acid sequence of AASSLQS (SEQ ID NO:71), and

a light chain CDR3 comprising the amino acid sequence of QQSYSTPPYT (SEQ ID NO:72);

(9) a light chain CDR1 comprising the amino acid sequence of RASQSIFNYVA (SEQ ID NO:73),

a light chain CDR2 comprising the amino acid sequence of DASNRAT (SEQ ID NO:74), and

a light chain CDR3 comprising the amino acid sequence of QQRSKWPPTWT (SEQ ID NO:75);

(10) a light chain CDR1 comprising the amino acid sequence of RASETVSSRQLA (SEQ ID NO:76),

a light chain CDR2 comprising the amino acid sequence of GASSRAT (SEQ ID NO:77), and

a light chain CDR3 comprising the amino acid sequence of QQYGSSPRT (SEQ ID NO:78);

(11) a light chain CDR1 comprising the amino acid sequence of RASQSVSSSSLA (SEQ ID NO:79),

a light chain CDR2 comprising the amino acid sequence of GASSRAT (SEQ ID NO:77), and

a light chain CDR3 comprising the amino acid sequence of QKYSSYPLT (SEQ ID NO:80); or

(12) a light chain CDR1 comprising the amino acid sequence of RASQSVGSNLA (SEQ ID NO:81),

a light chain CDR2 comprising the amino acid sequence of GASTGAT (SEQ ID NO:82), and

a light chain CDR3 comprising the amino acid sequence of QQYYSFLAKT (SEQ ID NO:83).

The recombinant proteins can further comprise a linker that links the fGCSF to the antigen binding fragment. In some embodiments, (i) a cysteine in the feline heavy chain constant 1 domain and/or (ii) a cysteine in the feline light chain constant domain that is/are located in an interchain disulfide bond between the light chain and the heavy chain is/are conserved, deleted, and/or substituted with an amino acid residue other than cysteine.

In some embodiments of the recombinant proteins disclosed herein, the heavy chain variable domain comprises a heavy chain CDR1 comprising the amino acid sequence of SEQ ID NO:64, a heavy chain CDR2 comprising the amino acid sequence of SEQ ID NO:65, and a heavy chain CDR3 comprising the amino acid sequence of SEQ ID NO:66, and the light chain variable domain comprises a light chain CDR1 comprising the amino acid sequence of SEQ ID NO:81, a light chain CDR2 comprising the amino acid sequence of SEQ ID NO:82, and a light chain CDR3 comprising the amino acid sequence of SEQ ID NO:83.

In some embodiments, the heavy chain variable domain comprises an amino acid sequence having at least 80% identity to SEQ ID NO:1, 2, 3, 4, 5, or 6.

In some embodiments, the light chain variable domain comprises an amino acid sequence having at least 80% identity to SEQ ID NO:7, 8, 9, 10, 11, 12, or 13.

In some embodiments, the heavy chain variable domain comprises the amino acid sequence of SEQ ID NO:1, 2, 3, 4, 5, or 6, and the light chain variable domain comprises the amino acid sequence of SEQ ID NO:7, 8, 9, 10, 11, 12, or 13.

In some embodiments, the feline heavy chain constant 1 domain comprises an amino acid sequence having at least 80% identity to SEQ ID NO:14. In some embodiments, the feline light chain constant domain comprises an amino acid sequence having at least 80% identity to SEQ ID NO:15.

In some embodiments, the fGCSF is modified by removing a free cysteine group and an O-sugar chain from a naturally occurring fGCSF. In some embodiments, the fGCSF comprises an amino acid sequence having at least 80% identity to SEQ ID NO:18. In some embodiments, the fGCSF comprises an amino acid sequence having at least 80% identity to SEQ ID NO:19. In some embodiments, the fGCSF comprises the amino acid sequence of SEQ ID NO:19.

In some embodiments, the linker links the fGCSF to a C-terminus of the feline heavy chain constant 1 domain, an N-terminus of the heavy chain variable domain, a C-terminus of the feline light chain constant domain, and/or an N-terminus of the light chain variable domain. In some embodiments, the linker comprises 1 to 50 amino acids or 1 to 20 amino acids. In some embodiments, the linker comprises a formula of (GpSs)n or (SpGs)n, wherein G is glycine, S is serine, p is an integer of 1 to 10, s is 0 or an integer of 1 to 10, p+s is an integer of 20 or less, and n is an integer of 1 to 20.

Disclosed herein are nucleic acid molecules encoding the recombinant proteins disclosed herein. Disclosed herein are expression vectors comprising the nucleic acid molecules disclosed herein. Disclosed herein are cells transformed with the expression vectors disclosed herein.

Disclosed herein are compositions comprising the recombinant proteins disclosed herein. Disclosed herein are pharmaceutical compositions comprising the compositions disclosed herein and a pharmaceutically acceptable excipient. Disclosed herein are kits comprising the compositions disclosed herein and labels comprising instructions for uses thereof.

Disclosed herein are methods of treating feline panleukopenia, comprising administering to subjects in need thereof the compositions disclosed herein. In some embodiments, the compositions increase white blood cells in blood of the subject. In some embodiments, the white blood cells are neutrophils, monocytes, basophils, or a combination thereof.

Also disclosed herein are uses of the compositions disclosed herein for the treatment of feline panleukopenia in subjects in need thereof. Also disclosed herein are the compositions disclosed herein for use in the treatment of feline panleukopenia in subjects in need thereof. Also disclosed herein are the use of the compositions disclosed herein for the manufacture of a medicament for treatment of feline panleukopenia in subjects in need thereof.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A and 1B show expression vectors of FL335, which is a chimeric antibody of a human anti-serum albumin Fab antibody fragment, wherein FIG. 1A shows a cleavage map of an FL335 Fd pd2535NT vector, and FIG. 1B shows a cleavage map of an APB-F1 L pd2539 vector,

FIG. 2 shows a cleavage map of a feline serum albumin pJK-dhfr vector, which is an expression vector of feline serum albumin;

FIG. 3 shows a cleavage map of a feline GCSF pd2535NT vector, which is an expression vector of natural fGCSF;

FIGS. 4A and 4B show the expression vectors of APB-F1, which is a fusion protein of FL335 and feline GCSF, wherein FIG. 4A shows a cleavage map of an APB-F1 (v1, v2) Fd pd2535NT vector, and FIG. 4B shows a cleavage map of an APB-F1 L pd2539 vector;

FIG. 5 shows a schematic illustration of the structure of FL335;

FIGS. 6A and 6B show the identification of FL335 Fd, L, and feline serum albumin in culture media after expression and purification processes according to one exemplary embodiment, wherein FIG. 6A shows SDS-PAGE results of identifying FL335, and FIG. 6B shows SDS-PAGE results of identifying feline serum albumin;

FIGS. 7A and 7B show the binding ability of FL335 to serum albumin, wherein FIG. 7A shows ELISA results of identifying the binding ability of SL335 to human serum albumin and feline serum albumin, and FIG. 7B shows ELISA results of identifying the binding ability of FL335 to human serum albumin and feline serum albumin;

FIGS. 8A and 8B show the identification of natural fGCSF and mutant fGCSF in culture media after expression and purification processes according to one exemplary embodiment, wherein FIG. 8A shows SDS-PAGE results of identifying natural fGCSF, and FIG. 8B shows SDS-PAGE results of identifying mutant fGCSF;

FIG. 9 shows a schematic illustration of the structure of APB-F1;

FIGS. 10A and 10B show the identification of APB-F1 in culture media after expression and purification processes according to one exemplary embodiment, wherein FIG. 10A shows SDS-PAGE results of identifying APB-F1(v1), and FIG. 10B shows SDS-PAGE results of identifying APB-F1(v2);

FIGS. 11A and 11B show the purity of APB-F1 samples after expression and purification processes according to one exemplary embodiment, wherein FIG. 11A shows SEC-HPLC results of analyzing an APB-F1(v1) sample, and FIG. 11B shows SEC-HPLC results of analyzing an APB-F1(v2) sample;

FIG. 12 shows results of a proliferation assay for M-NFS60 cells, performed by using APB-F1;

FIGS. 13A and 13B show the molecular weight of APB-F1, examined by intact mass spectrometry, wherein FIG. 13A shows results of examining the mass of APB-F1 Fd and FIG. 13B shows results of examining the mass of APB-F1 L;

FIG. 14 shows LC-MS/MS results of analyzing the N-terminal sequence of APB-F1 Fd using MASCOT software;

FIG. 15 shows the results of pharmacokinetic evaluation of APB-F1 in cats;

FIG. 16 shows the results of pharmacodynamic evaluation of APB-F1 in cats, wherein white blood cell levels in blood were examined;

FIG. 17 shows the results of pharmacodynamic evaluation of APB-F1 in cats, wherein neutrophil levels in blood were examined;

FIG. 18 shows the results of pharmacodynamic evaluation of APB-F1 in cats, wherein monocyte levels in blood were examined:

FIG. 19 shows the results of pharmacodynamic evaluation of APB-F1 in cats, wherein basophil levels in blood were examined;

FIG. 20 shows the results of pharmacodynamic evaluation of APB-F1 in cats, wherein lymphocyte levels in blood were examined; and

FIG. 21 shows the results of pharmacodynamic evaluation of APB-F1 in cats, wherein eosinophil levels in blood were examined.

### DETAILED DESCRIPTION

### Terminology

As used herein, the terms “about” and “approximately,” when used to modify a numeric value or numeric range, indicate that deviations up to 10% above and up to 10% below the value or range remain within the intended meaning of the recited value or range.

As used herein, the term “preventing” means all of actions by which feline panleukopenia is restrained or occurrence thereof is retarded by administering the pharmaceutical composition.

As used herein, the term “treating” means all of actions by which symptoms of feline panleukopenia have taken a turn for the better, improved, eliminated, or been modified favorably by administering the compositions disclosed herein.

As used herein, the term “subject” refers to a subject in need of treatment of feline panleukopenia, and more specifically, it can refer to a feline animal, cat, or domestic cat, e.g., a pet cat.

“Feline panleukopenia,” which is a disease to be prevented or treated by the pharmaceutical composition, is a disease characterized by a marked decrease in white blood cells, with clinical symptoms such as bloody stools, diarrhea, severe dehydration, malnutrition, etc., and is one of lethal diseases of all feline species, because it is highly contagious and has a high mortality rate. The feline panleukopenia can be caused by infection with feline parvo virus (FPV). Although administration of granulocyte colony-stimulating factor (GCSF) is widely used as a method of treating the disease, there are therapeutic limitations due to production of anti-drug antibody (ADA), etc.

### Antibodies and Fragments Thereof

Disclosed herein are recombinant proteins comprising (a) an antigen binding fragment comprising a heavy chain and a light chain and (b) a feline granulocyte colony-stimulating factor (fGCSF),

wherein the heavy chain comprises a heavy chain variable domain and a feline heavy chain constant 1 domain, wherein the heavy chain variable domain comprises

(1) a heavy chain complementarity determining domain 1 (CDR1) comprising the amino acid sequence of SYGIS (SEQ ID NO:51),

a heavy chain complementarity determining domain 2 (CDR) comprising the amino acid sequence of WINTYSGGTKYAQKFQG (SEQ ID NO:52), and

a heavy chain complementarity determining domain 3 (CDR3) comprising the amino acid sequence of LGHCQRGICSDALDT (SEQ ID NO:53);

(2) a heavy chain CDR1 comprising the amino acid sequence of SYGIS (SEQ ID NO:51),

a heavy chain CDR2 comprising the amino acid sequence of RINTYNGNTGYAQRLQG (SEQ ID NO:54), and

a heavy chain CDR3 comprising the amino acid sequence of LGHCQRGICSDALDT (SEQ ID NO:53);

(3) a heavy chain CDR1 comprising the amino acid sequence of NYGIH (SEQ ID NO:55),

a heavy chain CDR2 comprising the amino acid sequence of SISYDGSNKYYADSVKG (SEQ ID NO:56), and

a heavy chain CDR3 comprising the amino acid sequence of DVHYYGSGSYYNAFDI (SEQ ID NO:57);

(4) a heavy chain CDR1 comprising the amino acid sequence of SYAMS (SEQ ID NO:58),

a heavy chain CDR2 comprising the amino acid sequence of VISHDGGFQYYADSVKG (SEQ ID NO:59), and

a heavy chain CDR3 comprising the amino acid sequence of AGWLRQYGMDV (SEQ ID NO:60);

(5) a heavy chain CDR1 comprising the amino acid sequence of AYWIA (SEQ ID NO:61),

a heavy chain CDR2 comprising the amino acid sequence of MIWPPDADARYSPSFQG (SEQ ID NO:62), and

a heavy chain CDR3 comprising the amino acid sequence of LYSGSYSP (SEQ ID NO:63); or

(6) a heavy chain CDR1 comprising the amino acid sequence of AYSMN (SEQ ID NO:64),

a heavy chain CDR2 comprising the amino acid sequence of SISSSGRYIHYADSVKG (SEQ ID NO:65), and

a heavy chain CDR3 comprising the amino acid sequence of ETVMAGKALDY (SEQ ID NO:66); and

wherein the light chain comprises a light chain variable domain and a feline light chain constant domain, wherein the light chain variable domain comprises

(7) a light chain CDR1 comprising the amino acid sequence of RASQSISRYLN (SEQ ID NO:67),

a light chain CDR2 comprising the amino acid sequence of GASRLES (SEQ ID NO:68), and

a light chain CDR3 comprising the amino acid sequence of QQSDSVPVT (SEQ ID NO:69);

(8) a light chain CDR1 comprising the amino acid sequence of RASQSISSYLN (SEQ ID NO:70),

a light chain CDR2 comprising the amino acid sequence of AASSLQS (SEQ ID NO:71), and

a light chain CDR3 comprising the amino acid sequence of QQSYSTPPYT (SEQ ID NO:72);

(9) a light chain CDR1 comprising the amino acid sequence of RASQSIFNYVA (SEQ ID NO:73),

a light chain CDR2 comprising the amino acid sequence of DASNRAT (SEQ ID NO:74), and

a light chain CDR3 comprising the amino acid sequence of QQRSKWPPTWT (SEQ ID NO:75);

(10) alight chain CDR1 comprising the amino acid sequence of RASETVSSRQLA (SEQ ID NO:76),

a light chain CDR2 comprising the amino acid sequence of GASSRAT (SEQ ID NO:77), and

a light chain CDR3 comprising the amino acid sequence of QQYGSSPRT (SEQ ID NO:78);

(11) a light chain CDR1 comprising the amino acid sequence of RASQSVSSSSLA (SEQ ID NO:79),

a light chain CDR2 comprising the amino acid sequence of GASSRAT (SEQ ID NO:77), and

a light chain CDR3 comprising the amino acid sequence of QKYSSYPLT (SEQ ID NO:80); or

(12) a light chain CDR1 comprising the amino acid sequence of RASQSVGSNLA (SEQ ID NO:81),

a light chain CDR2 comprising the amino acid sequence of GASTGAT (SEQ ID NO:82), and

a light chain CDR3 comprising the amino acid sequence of QQYYSFLAKT (SEQ ID NO:83).

The recombinant proteins can further comprise a linker that links the fGCSF to the antigen binding fragment. In some embodiments, (i) a cysteine in the feline heavy chain constant 1 domain and/or (ii) a cysteine in the feline light chain constant domain that is/are located in an interchain disulfide bond between the light chain and the heavy chain is/are conserved, deleted, and/or substituted with an amino acid residue other than cysteine.

In some embodiments of the recombinant proteins disclosed herein, the heavy chain variable domain comprises a heavy chain CDR1 comprising the amino acid sequence of SEQ ID NO:64, a heavy chain CDR2 comprising the amino acid sequence of SEQ ID NO:65, and a heavy chain CDR3 comprising the amino acid sequence of SEQ ID NO:66, and the light chain variable domain comprises a light chain CDR1 comprising the amino acid sequence of SEQ ID NO:81, a light chain CDR2 comprising the amino acid sequence of SEQ ID NO:82, and a light chain CDR3 comprising the amino acid sequence of SEQ ID NO:83.

In some embodiments, the heavy chain variable domain comprises an amino acid sequence having at least 80%, at least 85%, at least 90%, at least 93%, at least 95%, at least 96%, at least 97%, at least 98%, at least 99%, or 100% identity to SEQ ID NO:1, 2, 3, 4, 5, or 6.

In some embodiments, the light chain variable domain comprises an amino acid sequence having at least 80%, at least 85%, at least 90%, at least 93%, at least 95%, at least 96%, at least 97%, at least 98%, at least 99%, or 100% identity to SEQ ID NO:7, 8, 9, 10, I1, 12, or 13.

In some embodiments, the heavy chain variable domain comprises the amino acid sequence of SEQ ID NO:1, 2, 3, 4, 5, or 6, and the light chain variable domain comprises the amino acid sequence of SEQ ID NO:7, 8, 9, 10, 11, 12, or 13.

In some embodiments, the feline heavy chain constant 1 domain comprises an amino acid sequence having at least 80%, at least 85%, at least 90%, at least 93%, at least 95%, at least 96%, at least 97%, at least 98%, at least 99%, or 100% identity to SEQ ID NO:14.

In some embodiments, the feline light chain constant domain comprises an amino acid sequence having at least 80%, at least 85%, at least 90%, at least 93%, at least 95%, at least 96%, at least 97%, at least 98%, at least 99%, or 100% identity to SEQ ID NO:15.

In some embodiments, the linker links the fGCSF to a C-terminus of the feline heavy chain constant 1 domain, an N-terminus of the heavy chain variable domain, a C-terminus of the feline light chain constant domain, and/or an N-terminus of the light chain variable domain. In some embodiments, the linker comprises 1 to 50 amino acids or 1 to 20 amino acids. In some embodiments, the linker comprises a formula of (GpSs)n or (SpGs)n, wherein G is glycine, S is serine, p is an integer of 1 to 10, s is 0 or an integer of 1 to 10, p+s is an integer of 20 or less, and n is an integer of 1 to 20.

As disclosed herein, recombinant proteins can be prepared by fusing feline GCSF with an FL355 Fab antibody fragment, which is a feline chimeric antibody fragment, and it was demonstrated that the recombinant proteins have improved pharmacokinetic properties and increases white blood cells to a therapeutically effective level in treatment of feline panleukopenia. Accordingly, disclosed herein are recombinant proteins including an antigen binding fragment binding to serum albumin; and a feline granulocyte colony-stimulating factor.

As used herein, the term “heavy chain (HC or CH)” refers to both a full length heavy chain and a fragment thereof, the full length heavy chain including a variable region domain VH including an amino acid sequence having a sufficient variable region (VR) sequence to confer specificity for an antigen and three constant region domains CH1, CH2, and CH3. As used herein, the term “light chain (LC or CL)” refers to both a full length light chain and a fragment thereof, the full length light chain including a variable region domain VL including an amino acid sequence having a sufficient VR sequence to confer specificity for an antigen and a constant region domain CL.

In some embodiments, the antigen binding fragment binding to albumin can be chimerized by including a heavy chain variable domain comprising an amino acid sequence of, e.g., SEQ ID NO:1, SEQ ID NO:2, SEQ ID NO:3, SEQ ID NO:4, SEQ ID NO:5, or SEQ ID NO:6 and a feline heavy chain constant 1 domain bound to the domain; and a light chain variable domain comprising an amino acid of, e.g., SEQ ID NO:7, SEQ ID NO:8, SEQ ID NO:9, SEQ ID NO:10. SEQ ID NO: 11, SEQ ID NO:12, or SEQ ID NO:13 and a feline light chain constant domain bound to the domain.

The feline heavy chain constant 1 domain and the feline light chain constant domain can be derived from an IgG1 antibody constant domain, and in any one or more thereof, cysteine which is an amino acid used in a disulfide bond between the light chain and the heavy chain domain can be conserved or deleted, or substituted with an amino acid residue other than cysteine. For example, the feline heavy chain constant 1 domain can comprise an amino acid sequence of SEQ ID NO:14, and the feline light chain constant domain can comprise an amino acid sequence of SEQ ID NO:15. The deletion or substitution of cysteine in the domain can contribute to improving an expression level of the recombinant protein in transformed cells during a process of producing the above mentioned recombinant protein. In some embodiments, (i) a cysteine in the feline heavy chain constant 1 domain and/or (ii) a cysteine in the feline light chain constant domain that is/are located in an interchain disulfide bond between the light chain and the heavy chain is/are conserved, deleted, and/or substituted with an amino acid residue other than cysteine.

In some embodiments, the feline chimeric antigen binding fragment binding can comprise a heavy chain comprising an amino acid sequence having at least 80%, at least 85%, at least 90%, at least 93%, at least 95%, at least 96%, at least 97%, at least 98%, at least 99%, or 100% identity to SEQ ID NO:16; and a light chain comprising an amino acid sequence having at least 80%, at least 85%, at least 90%, at least 93%, at least 95%, at least 96%, at least 97%, at least 98%, at least 99%, or 100% identity to SEQ ID NO:17.

The fGCSF can be a non-mutated natural protein, which can be obtained from a public database such as https://www.ncbi.nlm.nih.gov/, and e.g., it can comprise an amino acid sequence of SEQ ID NO:18, but is not limited thereto. In some embodiments, the feline granulocyte colony-stimulating factor can be modified by removing a free cysteine group and an O-sugar chain from the natural granulocyte colony-stimulating factor, and can comprise an amino acid sequence of SEQ ID NO:19, but is not limited thereto. The removal of the free cysteine group and the O-sugar chain can provide convenience of production, isolation, and purification processes of the recombinant proteins.

In some embodiments, the fGCSF comprises an amino acid sequence having at least 80%, at least 85%, at least 90%, at least 93%, at least 95%, at least 96%, at least 97%, at least 98%, at least 99%, or 100% identity to SEQ ID NO:18. In some embodiments, the fGCSF comprises an amino acid sequence having at least 80%, at least 85%, at least 90%, at least 93%, at least 95%, at least 96%, at least 97%, at least 98%, at least 99%, or 100% identity to SEQ ID NO:19. In some embodiments, the fGCSF comprises the amino acid sequence of SEQ ID NO:19.

In some embodiments, the antigen binding fragment binding to serum albumin and the feline granulocyte colony-stimulating factor can be linked to each other via a linker. For example, the linker can link the granulocyte colony-stimulating factor to any one region selected from the C-terminus of the heavy chain constant 1 domain, the N-terminus of the heavy chain variable domain, the C-terminus of the light chain constant domain, and the N-terminus of the light chain variable domain of the antigen binding fragment. Further, the linker can be appropriately modified for use, if needed. For example, the linker can be a polypeptide composed of 1 to 50 or 1 to 20 arbitrary or nonarbitrary amino acids. The peptide linker can include Gly, Asn, and Ser residues, and can also include neutral amino acids such as Thr and Ala. An amino acid sequence suitable for the peptide linker is known in the art. Adjusting the copy number “n” allows for optimization of the linker in order to achieve appropriate separation between the functional moieties or to maintain necessary inter-moiety interaction. Other linkers are known in the art, e.g., G and S linkers containing additional amino acid residues, such as T and A, to maintain flexibility, as well as polar amino acid residues to improve solubility. Therefore, the linker can be a flexible linker containing G. S, and/or T, A residues. The linker can have a general formula selected from (GpSs)ₙ and (SpGs)ₙ, wherein, independently, p is an integer of 1 to 10, s is 0 or an integer of 0 to 10, p+s is an integer of 20 or less, and n is an integer of 1 to 20. More specifically, examples of the linker can include (GGGGS)ₙ (SEQ ID NO:40), (SGGGG)ₙ (SEQ ID NO:41), (SRSSG)ₙ (SEQ ID NO:42), (SGSSC)ₙ (SEQ ID NO:43), (GKSSGSGSESKS)ₙ (SEQ ID NO:44), (RPPPPC)ₙ (SEQ ID NO:45), (SSPPPPC)ₙ (SEQ ID NO:46), (GSTSGSGKSSEGKG)ₙ (SEQ ID NO:47), (GSTSGSGKSSEGSGSTKG)ₙ (SEQ ID NO:48), (GSTSGSGKPGSGEGSTKG)ₙ (SEQ ID NO:49), or (EGKSSGSGSESKEF)ₙ (SEQ ID NO:50), wherein n can be an integer of 1 to 20, or 1 to 10.

In some embodiments, the linker links the fGCSF to a C-terminus of the feline heavy chain constant 1 domain, an N-terminus of the heavy chain variable domain, a C-terminus of the feline light chain constant domain, and/or an N-terminus of the light chain variable domain. In some embodiments, the linker comprises a formula of (GpSs)n or (SpGs)n, wherein G is glycine, S is serine, p is an integer of 1 to 10, s is 0 or an integer of 1 to 10, p+s is an integer of 20 or less, and n is an integer of 1 to 20. Further, the linker can comprise an amino acid sequence of SEQ ID NO:20 or SEQ ID NO:21, but is not limited thereto.

In some embodiments, the recombinant proteins can be composed of a combination of a heavy chain recombinant protein including an antigen binding fragment binding to feline serum albumin, wherein the antigen binding fragment is bound with a heavy chain variable domain and a feline heavy chain constant 1 domain, and a feline granulocyte colony-stimulating factor bound to the N-terminus of the feline heavy chain constant 1 domain; and an antigen binding fragment binding to feline serum albumin, wherein the antigen binding fragment is bound with a light chain variable domain and a feline light chain constant domain. Here, the heavy chain recombinant protein can comprise an amino acid sequence of at least 80%, at least 85%, at least 90%, at least 93%, at least 95%, at least 96%, at least 97%, at least 98%, at least 99%, or 100% identity to SEQ ID NO:22 or 23, and the antigen binding fragment including the light chain variable domain can comprise an amino acid sequence of at least 80%, at least 85%, at least 90%, at least 93%, at least 95%, at least 96%, at least 97%, at least 98%, at least 99%, or 100% identity to SEQ ID NO:17. The recombinant protein can have significantly improved pharmacokinetic properties while maintaining the intrinsic biological activity of the feline granulocyte colony-stimulating factor.

As used herein, the terms “antibody” and “antibodies” are terms of art and can be used interchangeably herein and refer to a molecule with an antigen-binding site that specifically binds an antigen. Antibodies can include, e.g., monoclonal antibodies, recombinantly produced antibodies, human antibodies, feline antibodies, resurfaced antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, single domain antibodies, monovalent antibodies, single chain antibodies or single-chain Fvs (scFv), camelized antibodies, affybodies, Fab fragments, F(ab′)₂ fragments, disulfide-linked Fvs (sdFv), anti-idiotypic (anti-Id) antibodies (including, e.g., anti-anti-Id antibodies), bispecific antibodies, and multispecific antibodies.

Antibodies can be of any type (e.g., IgG, IgE, IgM, IgD, IgA, or IgY), any class (e.g., IgG₁, IgG₂, IgG₃, IgG₄, IgA₁, or IgA₂), or any subclass (e.g., IgG₂ₐ or IgG_{2b}) of immunoglobulin molecule. In some embodiments, the antibody is a feline chimeric antibody.

As used herein, the terms “bioeffector moiety,” “antigen-binding domain,” “antigen-binding region,” “antigen-binding site,” and similar terms refer to the portions of the recombinant protein that comprises the amino acid residues that confer on the recombinant protein its specificity for the antigen (e.g., the complementarity determining regions (CDR)). The antigen-binding region can be derived from any animal species, such as feline, rodents (e.g., mouse, rat, or hamster) and humans.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody. The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody, or an antigen-binding portion thereof. In certain aspects, the CDRs of an antibody can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some embodiments, the CDRs of the antibodies described herein have been determined according to the Kabat numbering scheme.

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as BIAcore® or KinExA.

As used herein, a “conservative amino acid substitution” is one in which the amino acid residue is replaced with an amino acid residue having a similar side chain. Families of amino acid residues having side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In certain embodiments, one or more amino acid residues within a CDR(s) or within a framework region(s) of an antibody can be replaced with an amino acid residue with a similar side chain.

As used herein, an “epitope” is a term in the art and refers to a localized region of an antigen to which an antibody can specifically bind. An epitope can be, e.g., contiguous amino acids of a polypeptide (linear or contiguous epitope) or an epitope can, e.g., come together from two or more non-contiguous regions of a polypeptide or polypeptides (conformational, non-linear, discontinuous, or non-contiguous epitope). In certain embodiments, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta CrystallogrD Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques. In some embodiments, the epitope of an antibody is determined using alanine scanning mutagenesis studies.

As used herein, the terms “immunospecifically binds,” “immunospecifically recognizes,” “specifically binds,” and “specifically recognizes” are analogous terms in the context of antibodies and refer to molecules that bind to an antigen (e.g., epitope, immune complex, or binding partner of an antigen-binding site) as such binding is understood by one skilled in the art. For example, a molecule that specifically binds to an antigen can bind to other peptides or polypeptides, generally with lower affinity as determined by, e.g., immunoassays, BIAcore®, KinExA 3000 instrument (Sapidyne Instruments, Boise, Id.), or other assays known in the art. In some embodiments, molecules that immunospecifically bind to an antigen bind to the antigen with a K_{A} that is at least 2 logs, 2.5 logs, 3 logs, 4 logs or greater than the K_{A} when the molecules bind to another antigen.

In some embodiments, molecules that immunospecifically bind to an antigen do not cross react with other proteins under similar binding conditions. In some embodiments, molecules that immunospecifically bind to an antigen do not cross react with other proteins. In some embodiments, provided herein are recombinant proteins that bind to a specified antigen with higher affinity than to another unrelated antigen. In certain embodiments, provided herein is a recombinant protein that binds to a specified antigen (e.g., human serum albumin) with a 20%, 25%, 30%, 35%, 40%, 45%, 50%, 55%, 60%, 65%, 70%, 75%, 80%, 85%, 90%, 95% or higher affinity than to another, unrelated antigen as measured by, e.g., a radioimmunoassay, surface plasmon resonance, or kinetic exclusion assay. In some embodiments, the extent of binding of a recombinant protein described herein to an unrelated, protein is less than 10%, 15%, or 20% of the binding of the antibody to the specified antigen as measured by, e.g., a radioimmunoassay.

In some embodiments, provided herein are recombinant proteins that bind to an antigen of various species, such as feline, rodents (e.g., mouse, rat, or hamster) and humans. In some embodiments, provided herein are recombinant proteins that bind to a feline antigen with higher affinity than to another species of the antigen. In certain embodiments, provided herein are recombinant proteins that bind to a feline antigen with a 5%, 10%, 15%, 20%, 25%, 30%, 35%, 40%, 45%, 50%, 55%, 60%, 65%, 70% or higher affinity than to another species as measured by, e.g., a radioimmunoassay, surface plasmon resonance, or kinetic exclusion assay. In some embodiments, the recombinant proteins described herein, which bind to a feline antigen, will bind to another species of the antigen protein with less than 10%, 15%, or 20% of the binding of the antibody to the feline antigen protein as measured by, e.g., a radioimmunoassay, surface plasmon resonance, or kinetic exclusion assay.

As used herein, the term “host cell” can be any type of cell, e.g., a primary cell, a cell in culture, or a cell from a cell line. In embodiments, the term “host cell” refers to a cell transfected with a nucleic acid molecule and the progeny or potential progeny of such a cell. Progeny of such a cell cannot be identical to the parent cell transfected with the nucleic acid molecule, e.g., due to mutations or environmental influences that can occur in succeeding generations or integration of the nucleic acid molecule into the host cell genome.

As used herein, the term “effective amount” in the context of the administration of a therapy to a subject refers to the amount of a therapy that achieves a desired prophylactic or therapeutic effect.

In some embodiments, the Fab comprises a heavy chain variable domain comprising an amino acid sequence having at least 80%, at least 85%, at least 90%, at least 93%, at least 95%, at least 96%, at least 97%, at least 98%, at least 99%, or 100% identity to SEQ ID NO:1, 2, 3, 4, 5, or 6.

In some embodiments, the Fab comprises a light chain variable domain comprising an amino acid sequence having at least 80%, at least 85%, at least 90%, at least 93%, at least 95%, at least 96%, at least 97%, at least 98%, at least 99%, or 100% identity to SEQ ID NO:7, 8, 9, 10, 11, 12, or 13.

In some embodiments, the Fab comprises a heavy chain variable domain comprising an amino acid sequence having at least 80%, at least 85%, at least 90%, at least 93%, at least 95%, at least 96%, at least 97%, at least 98%, at least 99%, or 100% identity to SEQ ID NO: 1, 2, 3, 4, 5, or 6, and a light chain variable domain comprising an amino acid sequence having at least 80%, at least 85%, at least 90%, at least 93%, at least 95%, at least 96%, at least 97%, at least 98%, at least 99%, or 100% identity to SEQ ID NO: 7, 8, 9, 10, 11, or 12 or 13, respectively, or any combinations of heavy chain variable domain and light chain variable domain disclosed herein. For example, the Fab can comprise a heavy chain variable domain comprising an amino acid sequence having at least 80%, at least 85%, at least 90%, at least 93%, at least 95%, at least 96%, at least 97%, at least 98%, at least 99%, or 100% identity to SEQ ID NO:6 and a light chain variable domain comprising an amino acid sequence having at least 80%, at least 85%, at least 90%, at least 93%, at least 95%, at least 96%, at least 97%, at least 98%, at least 99%, or 100% identity to SEQ ID NO:13.

In some embodiments, the Fab comprises a heavy chain domain comprising an amino acid sequence having at least 80%, at least 85%, at least 90%, at least 93%, at least 95%, at least 96%, at least 97%, at least 98%, at least 99%, or 100% identity to SEQ ID NO:16 (V_{H}-C_{H1} domain) and a light chain domain comprising an amino acid sequence having at least 80%, at least 85%, at least 90%, at least 93%, at least 95%, at least 96%, at least 97%, at least 98%, at least 99%, or 100% identity to SEQ ID NO:17 (V_{L}-C_{L} domain).

In certain aspects, a recombinant protein described herein can be described by its VL domain alone, or its VH domain alone, or by its 3 VL CDRs alone, or its 3 VH CDRs alone. See, e.g., Rader C et al., (1998) PNAS 95: 8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In certain aspects, the CDRs of an antibody can be determined according to the Chothia numbering scheme, which refers to the location of immunoglobulin structural loops (see, e.g., Chothia C & Lesk A M, (1987), J Mol Biol 196: 901-917; Al-Lazikani B et al., (1997) J Mol Biol 273: 927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, provided herein are recombinant proteins that specifically bind to serum albumin (e.g., feline serum albumin) and comprise the Chothia VL CDRs of a VL. In certain aspects, provided herein are antibodies that specifically bind to serum albumin (e.g., human serum albumin) and comprise the Chothia VH CDRs of a VH. In certain aspects, provided herein are antibodies that specifically bind to serum albumin (e.g., human serum albumin) and comprise the Chothia VL CDRs of a VL and comprise the Chothia VH CDRs of a VH. In certain embodiments, antibodies that specifically bind to serum albumin (e.g., human serum albumin) comprise one or more CDRs, in which the Chothia and Kabat CDRs have the same amino acid sequence. In certain embodiments, provided herein are antibodies that specifically bind to serum albumin and comprise combinations of Kabat CDRs and Chothia CDRs.

In certain aspects, the CDRs of an antibody can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In certain aspects, the CDRs of an antibody can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an antibody can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.).

In some embodiments, the position of one or more CDRs along the VH (e.g., CDR1, CDR2, or CDR3) and/or VL (e.g., CDR1, CDR2, or CDR3) region of an antibody described herein can vary by one, two, three, four, five, or six amino acid positions so long as immunospecific binding to an antigen is maintained (e.g., substantially maintained, e.g., at least 50%, at least 60%, at least 70%, at least 80%, at least 90%6, at least 95%). For example, the position defining a CDR of an antibody described herein can vary by shifting the N-terminal and/or C-terminal boundary of the CDR by one, two, three, four, five, or six amino acids, relative to the CDR position of an antibody described herein, so long as immunospecific binding to the antigen(s) is maintained (e.g., substantially maintained, e.g., at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of an antibody described herein can vary (e.g., be shorter or longer) by one, two, three, four, five, or more amino acids, so long as immunospecific binding to the antigen(s) is maintained (e.g., substantially maintained, e.g., at least 50%, at least 60%, at least 70%, at least 80%, at least 90%, at least 95%).

In some embodiments, a VL CDR1, VL CDR2, VL CDR3, VH CDR1, VH CDR2, and/or VH CDR3 described herein can be one, two, three, four, five or more amino acids shorter than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, a VL CDR1, VL CDR2, VL CDR3, VH CDR1, VH CDR2, and/or VH CDR3 described herein can be one, two, three, four, five or more amino acids longer than one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the carboxy terminus of a VL CDR1, VL CDR2, VL CDR3, VH CDR1, VH CDR2, and/or VH CDR3 described herein can be extended by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In other embodiments, the amino terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 60%, at least 70%, at least 80%, at least 90%, at least 95%). In some embodiments, the carboxy terminus of a VL CDR1, VL CDR2, VL CDR3, VH CDR1, VH CDR2, and/or VH CDR3 described herein can be shortened by one, two, three, four, five or more amino acids compared to one or more of the CDRs described herein so long as immunospecific binding to the antigen(s) is maintained (e.g., substantially maintained, e.g., at least 50%, at least 600%, at least 70%, at least 80%, at least 90° %, at least 95%). Any method known in the art can be used to ascertain whether immunospecific binding to the antigen(s) is maintained, e.g., the binding assays and conditions described in the “Examples” section herein.

The determination of percent identity between two sequences (e.g., amino acid sequences or nucleic acid sequences) can also be accomplished using a mathematical algorithm. A specific, non-limiting example of a mathematical algorithm utilized for the comparison of two sequences is the algorithm of Karlin S & Altschul S F (1990) PNAS 87: 2264-2268, modified as in Karlin S & Altschul S F (1993) PNAS 90: 5873-5877. Such an algorithm is incorporated into the NBLAST and XBLAST programs of Altschul S F et al., (1990) J Mol Biol 215: 403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul S F et al., (1997) Nuc Acids Res 25: 3389 3402. Alternatively, PSI BLAST can be used to perform an iterated search which detects distant relationships between molecules (Id). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, nonlimiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

The recombinant proteins disclosed herein can be fused or conjugated (e.g., covalently or noncovalently linked) to a detectable label or substance. Examples of detectable labels or substances include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin. Such labeled antibodies can be used to detect antigen proteins.

### Antibody Production

According to one exemplary embodiment, a recombinant protein (APB-F1) was prepared, the recombinant protein (APB-F1) including an antigen binding fragment binding to feline serum albumin, wherein the antigen binding fragment is bound with a feline heavy chain constant 1 domain and a light chain constant domain; and a mutant feline granulocyte colony-stimulating factor fused to the feline heavy chain constant 1 domain. It was confirmed that the recombinant protein was obtained in a high yield while maintaining biological activities possessed by the respective factors.

Still other aspects provide methods of preparing the recombinant protein, the methods including (a) culturing the cells; and (b) recovering the recombinant protein from the cultured cells. The cells can be cultured in various media. A commercially available medium can be used as a culture medium without limitation. All other essential supplements known to those skilled in the art can also be included at appropriate concentrations. Culture conditions, e.g., temperature, pH, etc., are those previously used together with the host cell selected for expression, and will be apparent to those skilled in the art. The recovering of the recombinant proteins can be performed by removing impurities by, e.g., centrifugation or ultrafiltration, and purifying the resultant by, e.g., affinity chromatography, etc. Other additional purification techniques, e.g., anion or cation exchange chromatography, hydrophobic interaction chromatography, hydroxylapatite chromatography, etc. can be used.

Recombinant proteins disclosed herein can be produced by any method known in the art for the synthesis of antibodies, e.g., by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, e.g., in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some embodiments, the recombinant proteins described herein are antibodies (e.g., recombinant antibodies) prepared, expressed, created or isolated by any means that involves creation, e.g., via synthesis, genetic engineering of DNA sequences. In certain embodiments, such antibodies comprise sequences (e.g., DNA sequences or amino acid sequences) that do not naturally exist within the antibody germline repertoire of an animal or mammal (e.g., human) in vivo.

In some aspects, provided herein are methods of making recombinant proteins disclosed herein comprising culturing a cell or host cell described herein. In some aspects, provided herein are methods of making a recombinant protein comprising expressing (e.g., recombinantly expressing) the antibodies using a cell or host cell described herein (e.g., a cell or a host cell comprising polynucleotides encoding an antibody described herein). In some embodiments, the cell is an isolated cell. In some embodiments, the exogenous polynucleotides have been introduced into the cell. In some embodiments, the method further comprises purifying the antibody obtained from the cell or host cell.

Antibodies can be prepared using a wide variety of techniques known in the art including the use of hybridoma, recombinant, and phage display technologies, or a combination thereof. For example, monoclonal antibodies can be produced using hybridoma techniques including those known in the art and taught, e.g., in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced recombinantly from host cells exogenously expressing an antibody described herein.

A “monoclonal antibody,” as used herein, is an antibody produced by a single cell (e.g., hybridoma or host cell producing a recombinant antibody), wherein the antibody immunospecifically binds to an antigen (e.g., human serum albumin) as determined, e.g., by ELISA or other antigen-binding or competitive binding assay known in the art or in the Examples provided herein. In particular embodiments, a monoclonal antibody can be a chimeric antibody or a humanized antibody. In certain embodiments, a monoclonal antibody is a monovalent antibody or multivalent (e.g., bivalent) antibody. In certain embodiments, a monoclonal antibody can be a Fab fragment or a F(ab′)₂ fragment. Monoclonal antibodies described herein can, e.g., be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies expressed thereby are well known in the art (see, e.g., Chapter 11 in: Short Protocols in Molecular Biology, (2002)5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the antigen (e.g., human serum albumin)) used for immunization. Alternatively, lymphocytes can be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986)). Additionally, a RIMMS (repetitive immunization multiple sites) technique can be used to immunize an animal (Kilpatrick K E et al., (1997) Hybridoma 16:381-9, incorporated by reference in its entirety).

Antibodies described herein can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of a tetrameric antibody molecule and contains the complete light chain paired with the VH and CH1 domains of the heavy chain. A F(ab′)₂ fragment contains the two antigen-binding arms of a tetrameric antibody molecule linked by disulfide bonds in the hinge region.

Further, the antibodies described herein can also be generated using various phage display methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT/GB91/001134; WO90/02809, WO91/10737, WO92/01047, WO92/18619, WO93/11236, WO95/15982, WO95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

As described in the above references, after phage selection, the antibody coding regions from the phage can be isolated and used to generate antibodies, including human antibodies, and expressed in any desired host, including mammalian cells, insect cells, plant cells, yeast, and bacteria, e.g., as described below. Techniques to recombinantly produce antibodies such as Fab, Fab′ and F(ab′)₂ fragments can also be employed using methods known in the art such as those disclosed in WO92/22324; Mullinax R L et al., (1992) BioTechniques 12(6): 864-9; Sawai H et al., (1995) Am J Reprod Immunol 34: 26-34; and Better M et al., (1988) Science 240: 1041-1043.

In some aspects, to generate antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express antibodies, e.g., IgG, using techniques known to those of skill in the art.

A chimeric antibody is a molecule in which different portions of the antibody are derived from different immunoglobulin molecules. For example, a chimeric antibody can contain a variable region of a human monoclonal antibody fused to a constant region of a feline antibody. Methods for producing chimeric antibodies are known in the art. See, e.g., Morrison S L (1985) Science 229: 1202-7; Oi V T & Morrison S L (1986) BioTechniques 4: 214-221; Gillies S D et al., (1989) J Immunol Methods 125: 191-202; and U.S. Pat. Nos. 5,807,715, 4,816,567, 4,816,397, and 6,331,415.

A feline chimeric antibody is capable of binding to a predetermined antigen and which comprises a framework region having substantially the amino acid sequence of a feline immunoglobulin and CDRs having substantially the amino acid sequence of a human immunoglobulin.

### Polynucleotides, Vectors, and Cells

Disclosed herein are nucleic acid molecules encoding the recombinant proteins disclosed herein.

Disclosed herein are expression vectors comprising the nucleic acid molecules disclosed herein.

Disclosed herein are cells transformed with the expression vectors disclosed herein.

Since the nucleic acid, the expression vector, and the transformed cell include the above-described recombinant protein or the nucleic acid encoding the recombinant protein as it is, or they use the same, descriptions common thereto will be omitted.

For example, in some aspects, the recombinant protein can be produced by isolating the nucleic acid encoding the recombinant protein. The nucleic acid is isolated and inserted into a replicable vector to perform additional cloning (DNA amplification) or additional expression. On the basis of this, other aspects relate to a vector including the nucleic acid.

As used herein, the term “nucleic acid” comprehensively includes DNA (gDNA and cDNA) and RNA molecules, and nucleotides as basic units of the nucleic acid include not only natural nucleotides but also analogues having modified sugar or base moieties.

The nucleic acid is interpreted to include a nucleotide sequence showing substantial identity to the nucleotide sequence. Substantial identity means a nucleotide sequence showing at least 80% homology, more specifically at least 90% homology, and most specifically at least 95% homology, when the nucleotide sequence of the present disclosure and another optional sequence are aligned to correspond to each other as much as possible and the aligned sequences are analyzed using an algorithm commonly used in the art.

DNA encoding the recombinant protein is easily isolated or synthesized by using a common process (e.g., by using an oligonucleotide probe capable of specifically binding to the DNA encoding the recombinant protein). Many vectors are available. Vector components generally include, but are not limited to, one or more of the following: a signal sequence, an origin of replication, one or more marker genes, an enhancer element, a promoter, and a transcription termination sequence.

As used herein, the term “vector” includes, as a means to express a target gene in a host cell, plasmid vectors; cosmid vectors; viral vectors such as bacteriophage vectors, adenovirus vectors, retrovirus vectors, and adeno-associated virus vectors, etc. In the vector, the nucleic acid encoding the recombinant protein is operably linked to a promoter.

“Operably linked” refers to a functional linkage between a nucleic acid expression control sequence (e.g., a promoter, a signal sequence, an array of transcriptional regulatory factor binding sites) and another nucleic acid sequence, whereby the control sequence directs transcription and/or translation of another nucleic acid sequence.

When a prokaryotic cell is used as a host, a powerful promoter capable of directing transcription (e.g., tac promoter, lac promoter, lacUV5 promoter, lpp promoter, pLλ promoter, pRλ promoter, rac5 promoter, amp promoter, recA promoter, SP6 promoter, trp promoter and T7 promoter, etc.), a ribosome binding site for initiation of translation, and a transcription/translation termination sequence are generally included. For example, when a eukaryotic cell is used as a host, a promoter derived from the genome of a mammalian cell (e.g., metallothionein promoter, β-actin promoter, human hemoglobin promoter, and human muscle creatine promoter) or a promoter derived from mammalian viruses (e.g., adenovirus late promoter, vaccinia virus 7.5K promoter, SV40 promoter, cytomegalovirus (CMV) promoter, tk promoter of HSV, mouse mammary tumor virus (MMTV) promoter, LTR promoter of HIV, promoter of Moloney virus, promoter of Epstein-Barr virus (EBV), and promoter of Rous sarcoma virus (RSV)) can be used, and a polyadenylated sequence can be commonly used as the transcription termination sequence. In some cases, the vector can be fused with another sequence to facilitate purification of the recombinant protein expressed therefrom. The sequence to be fused includes, e.g., glutathione S-transferase (Pharmacia, USA), maltose binding protein (NEB, USA), FLAG (IBI, USA), 6× His (hexahistidine; Quiagen, USA), etc. The vector includes, as a selective marker, an antibiotic-resistant gene that is ordinarily used in the art, e.g., genes resistant against ampicillin, gentamycin, carbenicillin, chloramphenicol, streptomycin, kanamycin, geneticin, neomycin, and tetracycline.

In still other aspects, the present disclosure provides cells transformed with the above-mentioned vectors. The cells used to produce the recombinant protein of the present disclosure can be prokaryotic cells, yeast cells, or higher eukaryotic cells, but are not limited thereto. Prokaryotic host cells such as Escherichia coli, the genus bacillus strains such as Bacillus subtilis and Bacillus thuringiensis, Streptomyces, Pseudomonas (e.g., Pseudomonas putida), Proteus mirabilis and Staphylococcus (e.g., Staphylococcus carnosus) can be used. However, animal cells are most interested, and examples of the useful host cell line can include COS-7, BHK, CHO (GS null CHO-K1), CHOK1, DXB-11, DG-44, CHO/-DHFR, CV1, COS-7, HEK293, BHK, TM4, VERO, HELA, MDCK, BRL 3A, W138, Hep G2, SK-Hep, MMT, TRI, MRC 5, FS4, 3T3, RIN, A549, PC12, K562, PER.C6, SP2/0, NS-0, U20S, or HT1080, but are not limited thereto.

As used herein, the term “transformation” means a molecular biological technique that changes the genetic trait of a cell by a DNA chain fragment or plasmid which possesses a different type of foreign gene from that of the original cell, penetrates among the cells, and combines with DNA in the original cell. The transformation means insertion of the expression vector including the gene of the recombinant protein into a host cell.

Provided herein are nucleic acid molecules comprising a nucleotide sequence encoding a recombinant protein described herein (e.g., a variable light chain region and/or variable heavy chain region) that immunospecifically binds to an antigen, and vectors, e.g., vectors comprising such polynucleotides for recombinant expression in host cells (e.g., E. coli and mammalian cells). Provided herein are polynucleotides comprising nucleotide sequences encoding any of the antibodies provided herein, as well as vectors comprising such polynucleotide sequences, e.g., expression vectors for their efficient expression in host cells, e.g., mammalian cells.

As used herein, an “isolated” polynucleotide or nucleic acid molecule is one which is separated from other nucleic acid molecules which are present in the natural source (e.g., in a mouse or a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In some embodiments, a nucleic acid molecule(s) encoding an antibody described herein is isolated or purified.

Provided herein are polynucleotides comprising nucleotide sequences encoding antibodies, which immunospecifically bind to an antigen polypeptide (e.g., human serum albumin) and comprises an amino acid sequence as described herein, as well as antibodies that compete with such antibodies for binding to an antigen polypeptide (e.g., in a dose-dependent manner), or which binds to the same epitope as that of such antibodies.

Provided herein are polynucleotides comprising a nucleotide sequence encoding the light chain or heavy chain of an antibody described herein. The polynucleotides can comprise nucleotide sequences encoding a light chain comprising the VL FRs and CDRs of antibodies described herein. The polynucleotides can comprise nucleotide sequences encoding a heavy chain comprising the VH FRs and CDRs of antibodies described herein.

Provided herein are polynucleotides comprising a nucleotide sequence encoding a recombinant protein comprising a Fab comprising three VH chain CDRs, e.g., containing VL CDR1, VL CDR2, and VL CDR3 of an antibody to human serum albumin described herein and three VH chain CDRs, e.g., containing VH CDR1, VH CDR2, and VH CDR3 of an antibody to human serum albumin described herein.

Provided herein are polynucleotides comprising a nucleotide sequence encoding a recombinant protein comprising a VL domain.

In certain embodiments, a polynucleotide described herein comprises a nucleotide sequence encoding a recombinant protein provided herein comprising a light chain variable region comprising an amino acid sequence described herein (e.g., SEQ ID NO:7, 8, 9, 10, 11, 12, or 13), wherein the antibody immunospecifically binds to serum albumin.

In certain embodiments, a polynucleotide described herein comprises a nucleotide sequence encoding an antibody provided herein comprising a heavy chain variable region comprising an amino acid sequence described herein (e.g., SEQ ID NO:1, 2, 3, 4, 5, or 6), wherein the antibody immunospecifically binds to serum albumin.

In specific aspects, provided herein are polynucleotides comprising a nucleotide sequence encoding an antibody comprising a light chain and a heavy chain, e.g., a separate light chain and heavy chain. With respect to the light chain, in some embodiments, a polynucleotide provided herein comprises a nucleotide sequence encoding a kappa light chain. In other embodiments, a polynucleotide provided herein comprises a nucleotide sequence encoding a lambda light chain. In yet other embodiments, a polynucleotide provided herein comprises a nucleotide sequence encoding an antibody described herein comprising a human kappa light chain or a human lambda light chain. In some embodiments, a polynucleotide provided herein comprises a nucleotide sequence encoding an antibody, which immunospecifically binds to serum albumin, wherein the antibody comprises a light chain, and wherein the amino acid sequence of the VL domain can comprise the amino acid sequence set forth in SEQ ID NO:7, 8, 9, 10, 11, 12, or 13 and wherein the constant region of the light chain comprises the amino acid sequence of a feline kappa light chain constant region.

Also provided herein are polynucleotides encoding an antibody or a fragment thereof that are optimized, e.g., by codon/RNA optimization, replacement with heterologous signal sequences, and elimination of mRNA instability elements. Methods to generate optimized nucleic acids encoding an antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid.

In certain embodiments, an optimized polynucleotide sequence encoding an antibody described herein or a fragment thereof (e.g., VL domain or VH domain) can hybridize to an antisense (e.g., complementary) polynucleotide of an unoptimized polynucleotide sequence encoding an antibody described herein or a fragment thereof (e.g., VL domain or VH domain). In specific embodiments, an optimized nucleotide sequence encoding an antibody described herein or a fragment hybridizes under high stringency conditions to antisense polynucleotide of an unoptimized polynucleotide sequence encoding an antibody described herein or a fragment thereof. In some embodiments, an optimized nucleotide sequence encoding an antibody described herein or a fragment thereof hybridizes under high stringency, intermediate or lower stringency hybridization conditions to an antisense polynucleotide of an unoptimized nucleotide sequence encoding an antibody described herein or a fragment thereof. Information regarding hybridization conditions has been described, see, e.g., US 2005/0048549 (e.g., paragraphs 72-73), which is incorporated herein by reference.

The polynucleotides can be obtained, and the nucleotide sequence of the polynucleotides determined, by any method known in the art. Nucleotide sequences encoding antibodies described herein and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier G et al., (1994). BioTechniques 17: 242-246), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody or fragment thereof described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, e.g., to generate chimeric and humanized antibodies.

If a clone containing a nucleic acid encoding a particular antibody or fragment thereof is not available, but the sequence of the antibody molecule or fragment thereof is known, a nucleic acid encoding the immunoglobulin or fragment can be chemically synthesized or obtained from a suitable source (e.g., an antibody cDNA library or a cDNA library generated from, or nucleic acid, such as poly A+ RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding recombinant proteins described herein can be readily isolated and sequenced using conventional procedures (e.g., by using oligonucleotide probes that are capable of binding specifically to genes encoding the heavy and light chains of the recombinant proteins). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of recombinant proteins in the recombinant host cells.

To generate antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VII domains can be cloned into vectors expressing a heavy chain constant region, e.g., the human gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, e.g., by substituting the coding sequence for human heavy and light chain constant domains in place of the murine sequences, or by covalently joining to the immunoglobulin coding sequence all or part of the coding sequence for a non-immunoglobulin polypeptide.

Also provided are polynucleotides that hybridize under high stringency, intermediate or lower stringency hybridization conditions to polynucleotides that encode an antibody described herein. In specific embodiments, polynucleotides described herein hybridize under high stringency, intermediate or lower stringency hybridization conditions to polynucleotides encoding a VH domain and/or VL domain provided herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions are known to those of skill in the art and have been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3.

Further disclosed herein are expression vectors comprising:

(a) a promoter,

(b) a first nucleic acid molecule encoding an antigen binding fragment (Fab) that binds to serum albumin, and

(c) a second nucleic acid molecule encoding a bioactive effector moiety such as fGCSF and a linker,

wherein the promoter, the first nucleic acid sequence, and the second nucleic acid molecules are operably linked. The second nucleic acid molecule can encode 2, 3, 4, 5, 6, or more bioactive effector moieties and linkers.

Also disclosed herein are expression vectors comprising:

(a) a promoter and

(b) a nucleic acid molecule encoding a heavy chain variable domain as disclosed herein and a feline heavy chain constant 1 domain as disclosed herein.

Also disclosed herein are expression vectors comprising:

(a) a promoter and

(b) a nucleic acid molecule encoding a fGCSF as disclosed herein, a heavy chain variable domain as disclosed herein, and a feline heavy chain constant 1 domain as disclosed herein.

Also disclosed herein are expression vectors comprising:

(a) a promoter and

(b) a nucleic acid molecule encoding a light chain variable domain as disclosed herein and a feline light chain constant domain as disclosed herein.

Also disclosed herein are expression vectors comprising:

(a) a promoter and

(b) a nucleic acid molecule encoding a fGCSF as disclosed herein, a light chain variable domain as disclosed herein, and a feline light chain constant domain as disclosed herein. One, two, three, or more expression vectors or nucleic acid molecules can be expressed to produce the desired recombinant proteins.

In some embodiments, the first nucleic acid molecule or vector comprises a nucleic acid sequence encoding a recombinant protein comprising (a) an antigen binding fragment comprising a heavy chain, wherein the heavy chain comprises a heavy chain variable domain and a feline heavy chain constant 1 domain, wherein the heavy chain variable domain comprises

(1) a heavy chain variable domain comprising a heavy chain complementarity determining domain 1 (CDR1) comprising the amino acid sequence of SYGIS (SEQ ID NO:51),

a heavy chain complementarity determining domain 2 (CDR) comprising the amino acid sequence of WINTYSGGTKYAQKFQG (SEQ ID NO:52), and

a heavy chain complementarity determining domain 3 (CDR3) comprising the amino acid sequence of LGHCQRGICSDALDT (SEQ ID NO:53);

(2) a heavy chain CDR1 comprising the amino acid sequence of SYGIS (SEQ ID NO:51),

a heavy chain CDR2 comprising the amino acid sequence of RINTYNGNTGYAQRLQG (SEQ ID NO:54), and

a heavy chain CDR3 comprising the amino acid sequence of LGHCQRGICSDALDT (SEQ ID NO:53);

(3) a heavy chain CDR1 comprising the amino acid sequence of NYGIH (SEQ ID NO:55),

a heavy chain CDR2 comprising the amino acid sequence of SISYDGSNKYYADSVKG (SEQ ID NO:56), and

a heavy chain CDR3 comprising the amino acid sequence of DVHYYGSGSYYNAFDI (SEQ ID NO:57);

(4) a heavy chain CDR1 comprising the amino acid sequence of SYAMS (SEQ ID NO:58),

a heavy chain CDR2 comprising the amino acid sequence of VISHDGGFQYYADSVKG (SEQ ID NO:59), and

a heavy chain CDR3 comprising the amino acid sequence of AGWLRQYGMDV (SEQ ID NO:60);

(5) a heavy chain CDRlcomprising the amino acid sequence of AYWIA (SEQ ID NO:61),

a heavy chain CDR2 comprising the amino acid sequence of MIWPPDADARYSPSFQG (SEQ ID NO:62), and

a heavy chain CDR3 comprising the amino acid sequence of LYSGSYSP (SEQ ID NO:63); or

(6) a heavy chain CDR1 comprising the amino acid sequence of AYSMN (SEQ ID NO:64),

a heavy chain CDR2 comprising the amino acid sequence of SISSSGRYIHYADSVKG (SEQ ID NO:65), and

a heavy chain CDR3 comprising the amino acid sequence of ETVMAGKALDY (SEQ ID NO:66).

In some embodiments, the second nucleic acid molecule or vector comprises a nucleic acid sequence encoding a recombinant protein comprising (a) an antigen binding fragment comprising a light chain, wherein the light chain comprises a light chain variable domain and a feline light chain constant domain, wherein the light chain variable domain comprises

(7) a light chain CDR1 comprising the amino acid sequence of RASQSISRYLN (SEQ ID NO:67),

a light chain CDR2 comprising the amino acid sequence of GASRLES (SEQ ID NO:68), and

a light chain CDR3 comprising the amino acid sequence of QQSDSVPVT (SEQ ID NO:69);

(8) a light chain CDR1 comprising the amino acid sequence of RASQSISSYLN (SEQ ID NO:70),

a light chain CDR2 comprising the amino acid sequence of AASSLQS (SEQ ID NO:71), and

a light chain CDR3 comprising the amino acid sequence of QQSYSTPPYT (SEQ ID NO:72);

(9) a light chain CDR1 comprising the amino acid sequence of RASQSIFNYVA (SEQ ID NO:73).

a light chain CDR2 comprising the amino acid sequence of DASNRAT (SEQ TD NO:74), and

a light chain CDR3 comprising the amino acid sequence of QQRSKWPPTWT (SEQ ID NO:75);

(10) a light chain CDR1 comprising the amino acid sequence of RASETVSSRQLA (SEQ ID NO:76),

a light chain CDR2 comprising the amino acid sequence of GASSRAT (SEQ ID NO:77), and

a light chain CDR3 comprising the amino acid sequence of QQYGSSPRT (SEQ ID NO:78),

(11) alight chain CDR1 comprising the amino acid sequence of RASQSVSSSSLA (SEQ ID NO:79),

a light chain CDR2 comprising the amino acid sequence of GASSRAT (SEQ ID NO:77), and

a light chain CDR3 comprising the amino acid sequence of QKYSSYPLT (SEQ ID NO:80); or

(12) a light chain CDR1 comprising the amino acid sequence of RASQSVGSNLA (SEQ ID NO:81),

a light chain CDR2 comprising the amino acid sequence of GASTGAT (SEQ ID NO:82), and

a light chain CDR3 comprising the amino acid sequence of QQYYSFLAKT (SEQ ID NO:83).

For example, the nucleic acid molecule encoding fGCSF can be linked to the first or second nucleic acid molecule or vector described above.

In other embodiments, the first nucleic acid molecule can comprise a nucleic acid sequence encoding a Fab comprising: a heavy chain variable domain comprising (1) above and a light chain variable domain comprising (7) above; a heavy chain variable domain comprising (2) above and a light chain variable domain comprising (8) above; a heavy chain variable domain comprising (3) above and a light chain variable domain comprising (9) above; a heavy chain variable domain comprising (4) above and a light chain variable domain comprising (10) above; a heavy chain variable domain comprising (5) above and a light chain variable domain comprising (11) above; a heavy chain variable domain comprising (6) above and a light chain variable domain comprising (12) above; or any or all combinations of a heavy chain variable domain and a light chain variable domain described above. In some embodiments, the first nucleic acid molecule comprises a nucleic acid sequence encoding a Fab (FL335) comprising the heavy chain variable domain comprises a heavy chain CDR1 comprising the amino acid sequence of SEQ ID NO:64, a heavy chain CDR2 comprising the amino acid sequence of SEQ ID NO:65, and a heavy chain CDR3 comprising the amino acid sequence of SEQ ID NO:66, and the light chain variable domain comprises a light chain CDR1 comprising the amino acid sequence of SEQ ID NO:81, a light chain CDR2 comprising the amino acid sequence of SEQ ID NO:82, and a light chain CDR3 comprising the amino acid sequence of SEQ ID NO:83. The second nucleic acid molecule can encode the fGCSF.

In other embodiments, the first nucleic acid molecule or vector comprises a nucleic acid sequence encoding a Fab comprising a heavy chain variable domain comprising an amino acid sequence having at least 80%, at least 85%, at least 90%, at least 93%, at least 95%, at least 96%, at least 97%, at least 98%, at least 99%, or 100% identity to SEQ ID NO:1, 2, 3, 4, 5, or 6. In some embodiments, the second nucleic acid molecule or vector comprises a nucleic acid sequence encoding a Fab comprising a light chain variable domain comprising an amino acid sequence having at least 80%, at least 85%, at least 90%, at least 93%, at least 95%, at least 96%, at least 97%, at least 98%, at least 99%, or 100% identity to SEQ ID NO:6, 7, 8, 9, 10, 11, 12, or 13. The nucleic acid molecule encoding fGCSF can be linked to the first or second nucleic acid molecule or vector.

In some embodiments, the first nucleic acid molecule or vector comprises a nucleic acid sequence encoding a Fab comprising a heavy chain variable domain comprising an amino acid sequence having at least 80%, at least 85%, at least 90%, at least 93%, at least 95%, at least 96%, at least 97%, at least 98%, at least 99%, or 100% identity to SEQ ID NO: 1, 2, 3, 4, 5, or 6, and a light chain variable domain comprising an amino acid sequence having at least 80%, at least 85%, at least 90%, at least 93%, at least 95%, at least 96%, at least 97%, at least 98%, at least 99%, or 100% identity to SEQ ID NO: 6, 7, 8, 9, 10, 11, or 12 or 13, respectively.

In some embodiments, the first nucleic acid molecule comprises a nucleic acid sequence encoding a Fab (SL335) comprising a heavy chain domain comprising an amino acid sequence of SEQ ID NO:16 (V_{H}-C_{H1} domain) and a light chain domain comprising an amino acid sequence of SEQ ID NO:17 (V_{L}-C_{L} domain).

In some embodiments, the bioactive effector moiety is fGCSF. For example, the second nucleic acid molecule can comprise a nucleotide sequence encoding the amino acid sequence having at least 80%, at least 85%, at least 90%, at least 93%, at least 95%, at least 96%, at least 97%, at least 98%, at least 99%, or 100% identity to one or more of SEQ ID NOS:18 and 19.

Recombinant expression of an antibody or fragment thereof described herein (e.g., a heavy or light chain of an antibody described herein) that specifically binds to involves construction of an expression vector containing a polynucleotide that encodes the antibody or fragment. Once a polynucleotide encoding an antibody or fragment thereof (e.g., heavy or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, e.g., in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, e.g., include the nucleotide sequence encoding the constant region of the antibody molecule (see, e.g., WO86/05807 and WO89/01036; and U.S. Pat. No. 5,122,464) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein.

A variety of host-expression vector systems can be utilized to express antibody molecules described. Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, and NIH 3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, cells for expressing antibodies described herein (e.g., an antibody comprising the CDRs of any one of antibodies pab1949 or pab2044) are CHO cells, e.g. CHO cells from the CHO GS System™ (Lonza). In some embodiments, cells for expressing antibodies described herein are human cells, e.g., human cell lines. In some embodiments, a mammalian expression vector is pOptiVEC™ or pcDNA3.3. In some embodiments, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45: 101-105; and Cockett M I et al., (1990) Biotechnology 8: 662-667). In certain embodiments, antibodies described herein are produced by CHO cells or NS0 cells. In some embodiments, the expression of nucleotide sequences encoding antibodies described herein is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the antibody coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan J & Shenk T (1984) PNAS 81: 3655-3659). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol 153: 516-544).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT2O and T47D, NS0 (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells. In certain embodiments, recombinant proteins described herein (e.g., an antibody comprising the CDRs are produced in mammalian cells, such as CHO cells.

In some embodiments, the antibodies described herein have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express recombinant proteins disclosed herein can be engineered. In specific embodiments, a cell provided herein stably expresses a light chain/light chain variable domain and a heavy chain/heavy chain variable domain which associate to form an antibody described herein (e.g., an antibody comprising the CDRs).

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an antibody described herein or a fragment thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11(1): 223-232), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-823) genes can be employed in tk-, hgprt- or aprt-cells, respectively. Also, antimetabolite resistance can be used as the basis of selection for the following genes: dhfr, which confers resistance to methotrexate (Wigler M et al., (1980) PNAS 77(6): 3567-3570; O'Hare K et al., (1981) PNAS 78: 1527-1531); gpt, which confers resistance to mycophenolic acid (Mulligan R C & Berg P (1981) PNAS 78(4): 2072-2076); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Felgner P L (1993) Trends Biotechnol 11(5): 211-215); and hygro, which confers resistance to hygromycin (Santerre R F et al., (1984) Gene 30(1-3): 147-156).

The expression levels of an antibody molecule can be increased by vector amplification (for a review, see Bebbington C R & Hentschel CCG, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse G F et al., (1983) Mol Cell Biol 3: 257-66).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1.15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322: 562-565; and Köhler G (1980) PNAS 77: 2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

The vector can comprise a first nucleic acid molecule encoding an antigen binding fragment (Fab) that bind to serum albumin, and a second nucleic acid molecule encoding a bioactive effector moiety and a linker.

Once an antibody molecule described herein has been produced by recombinant expression, it can be purified by any method known in the art for purification of an immunoglobulin molecule, e.g., by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in some embodiments, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, e.g., different post-translational modified forms of an antibody. When the antibody or fragment is recombinantly produced, it is also generally substantially free of culture medium, i.e., culture medium represents less than about 20%, 10%, 2%, 1%, 0.5%, or 0.1% of the volume of the protein preparation. When the antibody or fragment is produced by chemical synthesis, it is generally substantially free of chemical precursors or other chemicals, i.e., it is separated from chemical precursors or other chemicals which are involved in the synthesis of the protein. Accordingly, such preparations of the antibody or fragment have less than about 30%, 20%, 10/u, or 5% (by dry weight) of chemical precursors or compounds other than the antibody or fragment of interest. In some embodiments, antibodies described herein are isolated or purified.

### Compositions

Still other aspects provide compositions, e.g., pharmaceutical compositions, for preventing or treating feline panleukopenia, the pharmaceutical compositions comprising the recombinant protein as an active ingredient; a method of treating feline panleukopenia, the method comprising administering the composition to a subject; and medical use of the recombinant protein for preventing or treating feline panleukopenia.

For example, the pharmaceutical composition can comprise (a) a pharmaceutically effective amount of the recombinant protein; and (b) a pharmaceutically acceptable carrier.

In some embodiments, the in vivo half-life of the pharmaceutical composition can exhibit a 3- to 20-fold increase, as compared with that of feline GCSF. The in vivo half-life can exhibit, e.g., about 3.5-fold to about 6-fold increase, about 4-fold to about 6-fold increase, about 4.5-fold to about 6-fold increase, about 5-fold to about 6-fold increase, about 5.5-fold to about 6-fold increase, about 3-fold to about 5.5-fold increase, about 3.5-fold to about 5.5-fold increase, about 4-fold to about 5.5-fold increase, about 4.5-fold to about 5.5-fold increase, about 5-fold to about 5.5-fold increase, about 3-fold to about 5-fold increase, about 3.5-fold to about 5-fold increase, about 4-fold to about 5-fold increase, about 4.5-fold to about 5-fold increase, about 3-fold to about 4.5-fold increase, about 3.5-fold to about 4.5-fold increase, or about 4-fold to about 4.5-fold increase, as compared with that of feline GCSF. In some embodiments, addition, the in vivo half-life of the feline GCSF can be evaluated after subcutaneous injection of the mutant fGCSF.

In some embodiments, the pharmaceutical composition can increase white blood cell levels in blood. The white blood cells can be, e.g., neutrophils, monocytes, basophils, or a combination thereof. The increased white blood cell level can be sustained and maintained until day 20 after administration, day 15 after administration, day 12 after administration, day 10 after administration, day 8 after administration, or day 7 after administration.

The pharmaceutically acceptable carrier included in the pharmaceutical composition can include those commonly used in formulation, such as lactose, dextrose, sucrose, sorbitol, mannitol, starch, acacia rubber, calcium phosphate, alginate, gelatin, calcium silicate, microcrystalline cellulose, polyvinylpyrrolidone, water, syrup, methyl cellulose, methyl hydroxybenzoate, propyl hydroxybenzoate, talc, magnesium stearate, mineral oil, etc., but is not limited thereto. The composition of the present disclosure can further include a lubricant, a wetting agent, a sweetener, a flavoring agent, an emulsifying agent, a suspending agent, a preservative, etc., in addition to the above components.

The pharmaceutical composition can be orally or parenterally administered. Specifically, the pharmaceutical composition can be parenterally administered, and in this case, it can be administered by intravenous injection, subcutaneous injection, intramuscular injection, intraperitoneal injection, endothelial administration, topical administration, intranasal administration, intrapulmonary administration, and rectal administration. In some embodiments, it can be administered in the form of subcutaneous injection. When orally administered, a protein or peptide is digested, and therefore, it is required to formulate an oral composition by coating the active ingredient or protecting it from degradation in the stomach. In addition, the pharmaceutical composition can be administered by any device capable of delivering an active substance to target cells.

The pharmaceutical composition is administered in a pharmaceutically effective amount. As used herein, the “pharmaceutically effective amount” refers to an amount sufficient to treat a disease at a reasonable benefit/risk ratio applicable to any medical treatment. An effective dose level can be determined depending on factors including a patient's disease type, severity, drug activity, drug sensitivity, administration time, administration route and excretion ratio, treatment period, and co-administered drugs, and other factors well known in the medical field. The pharmaceutical composition can be administered as a single therapeutic agent or in combination with other therapeutic drugs, and can be administered with existing therapeutic drugs simultaneously, separately, or sequentially, once or in a few divided doses. It is important to administer the composition in a minimum amount sufficient to obtain the maximum effect without any side effects, considering all the factors, and this amount can be easily determined by those skilled in the art. As used herein, the term “pharmaceutically effective amount” refers to an amount sufficient to prevent or treat feline panleukopenia.

The pharmaceutical composition can be prepared in a unit dosage form or in a multi-dose container by formulating using a pharmaceutically acceptable carrier and/or excipient according to a method that can be easily carried out by a person skilled in the art to which the present disclosure pertains. In this case, the formulation can be in the form of a solution, suspension, or emulsion in an oily or aqueous medium, or in the form of an extract, a suppository, a powder, granules, a tablet, or a capsule, and the formulation can further include a dispersing agent or a stabilizing agent.

Provided herein are compositions comprising a recombinant protein described herein having the desired degree of purity in a physiologically acceptable carrier, excipient or stabilizer (Remington's Pharmaceutical Sciences (1990) Mack Publishing Co., Easton, Pa.). Also disclosed herein are pharmaceutical compositions comprising a recombinant protein described herein and a pharmaceutically acceptable excipient. Acceptable carriers, excipients, or stabilizers are nontoxic to recipients at the dosages and concentrations employed.

The pharmaceutical composition of the present disclosure can provide rapid, sustained or delayed release of an active ingredient after being administered to a subject and can be formulated using a method well known to those skilled in the art. The formulations can be in the form of a tablet, pill, powder, sachet, elixir, suspension, emulsion, solution, syrup, aerosol, soft or hard gelatin capsule, sterile injectable solution, sterile powder, or the like. Examples of suitable carriers, excipients, and diluents are lactose, dextrose, sucrose, sorbitol, mannitol, xylitol, erythritol, maltitol, starches, gum acacia, alginate, gelatin, calcium phosphate, calcium silicate, cellulose, microcrystalline cellulose, polyvinyl pyrrolidone, water, methylhydroxybenzoate, propylhydroxybenzoate, talc, magnesium stearate and mineral oil. Further, the formulations can additionally include a filler, an anti-agglutinating agent, a lubricating agent, a wetting agent, a favoring agent, an emulsifier, a preservative, and the like.

Pharmaceutical compositions described herein can be useful in enhancing, inducing, or activating the activities of the recombinant proteins disclosed herein and treating a disease or condition.

The compositions to be used for in vivo administration can be sterile. This is readily accomplished by filtration through, e.g., sterile filtration membranes.

### Uses and Methods

Also disclosed here are methods of treating feline panleukopenia in a subject in need thereof, the method comprising administering the pharmaceutical compositions disclosed herein to the subject. The subject is feline, e.g., domestic or wild cats, lions, tigers, jaguars, cheetahs, lynxes, etc.

Also disclosed herein are uses of the compositions disclosed herein for the treatment of feline panleukopenia in subjects in need thereof. Also disclosed herein are the compositions disclosed herein for use in the treatment of feline panleukopenia in subjects in need thereof. Also disclosed herein are the use of the compositions disclosed herein for the manufacture of a medicament for treatment of feline panleukopenia in subjects in need thereof.

Feline panleukopenia virus (FPLV) is a species of parvovirus that can infect wild and domestic members of the felid (cat) family worldwide. It is a highly contagious, severe infection that causes gastrointestinal, immune system, and nervous system disease. Its primary effect is to decrease the number of white blood cells.

In some embodiments, the compositions increase white blood cells in blood of the subject. In some embodiments, the white blood cells are neutrophils, monocytes, basophils, or a combination thereof.

An exemplary recombinant protein (APB-F1) prepared by fusing an FL355 Fab antibody fragment, which is a feline chimeric antibody fragment, with a feline granulocyte colony-stimulating factor, showed an in-vivo half-life of about 13.3 hr in a cat, which is about 4.9-fold increase, as compared with fGCSF showing an in-vivo half-life of 2.7 hr, and increased levels of lymphocytes and neutrophils in blood by administration of the recombinant protein were maintained until day 20 and day 11 after administration, respectively. Therefore, it was confirmed that the recombinant proteins disclosed herein can be used as an active ingredients of the pharmaceutical compositions for preventing or treating feline panleukopenia.

In some embodiments, an elimination half-life (T½) of the recombinant protein is at least about 2-fold greater, at least about 3-fold greater, at least about 4-fold greater, at least about 5-fold greater, at least about 7-fold greater, at least about 10-fold greater, or any folds or ranges of fold derived therefrom than that of a feline granulocyte colony-stimulating factor (fGCSF). In some embodiments, the recombinant protein has an elimination half-life (T½) of about 8 hrs to about 20 hrs, about 10 hrs to about 18 hours, about 12 hrs to about 15 hrs, or any half-life or ranges derived therefrom. In some embodiments, a Tmax of the recombinant protein is at least about 10% to about 200% higher, about 50% to 100% higher, about 50% to 75% higher, or any % or ranges of % derived therefrom greater than a Tmax of fGCSF. In some embodiments, a dose of the recombinant protein at about 360 ug/kg of the subject provides a Tmax of about 8 hrs to about 20 hrs, about 10 hrs to about 15 hrs, about 12 hrs to about 14 hrs, or any Tmax or ranges of Tmax derived therefrom. In some embodiments, a Cmax of the recombinant protein is at least about 10% higher, at least about 20% higher, at least about 30% higher, or any % or ranges of % derived therefrom than a Cmax of fGCSF. In some embodiments, a dose of the recombinant protein at about 360 ug/kg of the subject provides a Cmax of about 700 ng/ml to about 1000 ng/ml, about 750 ng/ml to about 900 ng/ml, about 800 ng/ml to about 850 ng/ml, or any doses or ranges of doses derived therefrom. In some embodiments, an AUClast of the recombinant protein is at least about 2-fold greater, at least 3-fold greater, at least 4-fold greater, at least 5-fold greater, or any fold or ranges of folds derived therefrom than an AUClast of a fGCSF. In some embodiments, a dose of the recombinant protein at about 360 ug/kg of the subject provides an AUClast of about 8000 hr*ng/ml to about 25000 hr*ng/ml, about 16000 hr*ng/ml to about 22000 hr*ng/ml, about 18000 hr*ng/ml to about 20000 hr*mg/ml, or any concentrations or ranges of concentrations derived therefrom.

In some aspects, presented herein are methods for modulating one or more immune functions or responses in a subject, comprising to a subject in need thereof administering an antibody described herein, or a composition thereof. Disclosed herein are methods for activating, enhancing or inducing one or more immune functions or responses in a subject, comprising to a subject in need thereof administering an antibody or a composition thereof. In some embodiments, presented herein are methods for preventing and/or treating diseases in which it is desirable to activate or enhance one or more immune functions or responses, comprising administering to a subject in need thereof an antibody described herein or a composition thereof. In certain embodiments, presented herein are methods of treating an autoimmune disease or condition comprising administering to a subject in need thereof an antibody or a composition thereof.

In some embodiments, an antibody described herein activates or enhances or induces one or more immune functions or responses in a subject by at least 99%, at least 98%, at least 95%, at least 90%, at least 85%, at least 80%, at least 75%, at least 70%, at least 60%, at least 50%, at least 45%, at least 40%, at least 45%, at least 35%, at least 30%, at least 25%, at least 20%, or at least 10%, or in the range of between 10% to 25%, 25% to 50%, 50% to 75%, or 75% to 95% relative to the immune function in a subject not administered the recombinant protein described herein using assays well known in the art, e.g., ELISPOT, ELISA, and cell proliferation assays.

### Routes of Administration & Dosages

The pharmaceutical compositions of the present disclosure can be administered to a subject through a variety of administration routes including oral, transcutaneous, subcutaneous, intravenous, and intramuscular administration routes.

The amount of a recombinant protein or composition disclosed herein that will be effective in the treatment and/or prevention of a condition will depend on the nature of the disease and can be determined by standard clinical techniques.

In the present disclosure, the amount of the recombinant protein disclosed herein that is actually administered is determined in light of various relevant factors including the disease to be treated, a selected route of administration, the age, sex and body weight of a patient, and severity of the disease, and the type of a bioactive polypeptide as an active ingredient. Since the recombinant protein of the present disclosure has excellent sustainability in blood, the number and frequency of administration of the peptide preparations comprising the recombinant protein of the present disclosure can be noticeably reduced.

The precise dose to be employed in a composition will also depend on the route of administration, and the seriousness of the disease, and should be decided according to the judgment of the practitioner and each subject's circumstances. For example, effective doses can also vary depending upon means of administration, target site, physiological state of the patient (including age, body weight and health), other medications administered, or whether treatment is prophylactic or therapeutic. Usually, the patient is a feline, domestic cat, or a pet cat. Treatment dosages are optimally titrated to optimize safety and efficacy.

In certain embodiments, an in vitro assay is employed to help identify optimal dosage ranges. Effective doses can be extrapolated from dose response curves derived from in vitro or animal model test systems.

### Kits

Provided herein are kits comprising one or more recombinant proteins described herein or conjugates thereof. Disclosed herein are kits comprising the compositions disclosed herein and labels comprising instructions for uses thereof. In some embodiments, provided herein is a pharmaceutical pack or kit comprising one or more containers filled with one or more of the ingredients of the pharmaceutical compositions described herein, such as one or more recombinant proteins provided herein. In some embodiments, the kits contain a pharmaceutical composition described herein and any prophylactic or therapeutic agent, such as those described herein. Optionally associated with such container(s) can be a notice in the form prescribed by a governmental agency regulating the manufacture, use or sale of pharmaceuticals or biological products, which notice reflects approval by the agency of manufacture, use or sale for feline administration. Also provided herein are kits that can be used in the above methods. In some embodiments, a kit comprises a recombinant protein described herein, e.g., a purified recombinant protein, in one or more containers. In some embodiments, kits described herein contain a substantially isolated antigen(s) (e.g., feline serum albumin) that can be used as a control. In other embodiments, the kits described herein further comprise a control antibody which does not react with a serum albumin antigen. In other embodiments, kits described herein contain one or more elements for detecting the binding of a recombinant protein to a serum albumin antigen (e.g., the recombinant protein can be conjugated to a detectable substrate such as a fluorescent compound, an enzymatic substrate, a radioactive compound or a luminescent compound, or a second antibody which recognizes the first antibody can be conjugated to a detectable substrate). In specific embodiments, a kit provided herein can include a recombinantly produced or chemically synthesized serum albumin antigen. The serum albumin antigen provided in the kit can also be attached to a solid support. In some embodiments, the detecting means of the above described kits include a solid support to which a serum albumin antigen is attached. Such kits can also include a non-attached reporter-labeled anti-feline antibody or anti-mouse/rat antibody. In binding of the antibody to the serum albumin, the antigen can be detected by binding of the said reporter-labeled antibody.

Hereinafter, the present disclosure will be described in more detail with reference to exemplary embodiments. However, these exemplary embodiments are only for illustrating the present disclosure, and the scope of the present disclosure is not limited thereto.

### EXAMPLES

### Preparation Example 1. Preparation of Expression Vector

All DNA cloning experiments were performed according to standard procedures. Feline IgG CH1(delta C, IMGT AB016710 IGHG1*01, Felis catus, CH1), Feline C L kappa (delta C, IMGT000050 IGKC*01, Felis catus, C-region), feline serum albumin (FSA, UniProtKB P49064), feline granulocyte colony-stimulating factor (fGCSF, UniProtKB Q9GJU0)), and APB-F1 (v2) (FL335Fd+mutant fGCSF) genes were obtained from Cosmogenetech Co., Ltd. (South Korea). Further, in polymerase chain reaction (PCR), pyrobest DNA polymerase (Takara, Japan) was used as a polymerase, and PCR was carried out using a T100 Thermal cycler (Bio-Rad, Hercules, Caligornia) machine. Further, the gene was inserted into an expression vector using a T4 DNA ligase (NEB Bio Lab, Ipswich, Mass.). Meanwhile, sequence information about primers is shown in Table 1 below.

TABLE 1

| | Oligonucleotide | SEQ |
|---|---|---|
| Primer | Sequence | ID |
| No. | (5'- 3') | NO: |
| 1 | gatcaactct agagccacca | 25 |
| | tggagtggtc ctgggtc | |
| 2 | tgagctgact gtgaccagag | 26 |
| | tg | |
| 3 | ggtcacagtc agctcagctt | 27 |
| | ctaccaccgc accttc | |
| 4 | aggaagacgc ttttagaggc | 28 |
| | ggccgctcag tcggttcggt | |
| | ccactgtttt atcgac | |
| 5 | gacatcgtcc tgacccagag | 29 |
| | ccccg | |
| 6 | ccgcttgatc tccagctggg | 30 |
| | tgcc | |
| 7 | cagctggaga tcaagcggag | 31 |
| | tgacgcacaa ccttctg | |
| 8 | aggaagacgc ttttagagct | 32 |
| | actccctctg ggactcgctc | |
| | cgattaaa | |
| 9 | accaccggag tgctttccac | 33 |
| | ccccctggga ccaacctcca | |
| | gcctgcccca gtcc | |
| 10 | atcggcggcc gcgaagacgc | 34 |
| | ttttagatca gtggtggtgg | |
| | tgatggtggt ggtgcccagg | |
| 11 | gcgtgaccac cggagtgctt | 35 |
| | tccgcaaccc cactgggacc | |
| | aaccagttc | |
| 12 | atcggcggcc gcgaagacgc | 36 |
| | ttttagatca gtggtggtgg | |
| | tgatggtggt ggtgccctgg | |
| | cttggtaaag tggcgaagag | |
| | cccg | |
| 13 | cttggagcca gaggcaagac | 37 |
| | agaag | |
| 14 | cttctgtctt gcctctggct | 38 |
| | ccaag | |
| 15 | aggaagacgc ttttagaggc | 39 |
| | ggccgctcaa gg | |

(1) Preparation of FL335 expression vector.

To prepare FL335, which is a chimeric antibody fragment of a human anti-serum albumin Fab antibody fragment SL335, amplification of V_{H} gene, which is a variable region of the SL335 Fab antibody fragment, was carried out by PCR using primers 1 and 2 under conditions of 30 cycles consisting of 5° C. for 30 sec, 60° C. for 20 sec, and 72° C. for 50 sec. Amplification of V_{L} gene was also carried out by PCR using primers 5 and 6 under the same conditions as above. Amplification of SL335 V_{H} gene and feline IgG CH₁ were carried out by linking PCR using primers 1 and 4 under conditions of 30 cycles consisting of 95° C. for 30 sec, 60° C. for 30 sec, and 72° C. for 1 min. As a result, a chimeric Fd (V_{H}+CH₁) gene was prepared. Amplification of SL335 V_{L} gene and feline light chain (kappa) C_{L} were also carried out by linking PCR using primers 5 and 8 under the same conditions as above. As a result, a chimeric L (V_{L}+C_{L}k) gene was obtained.

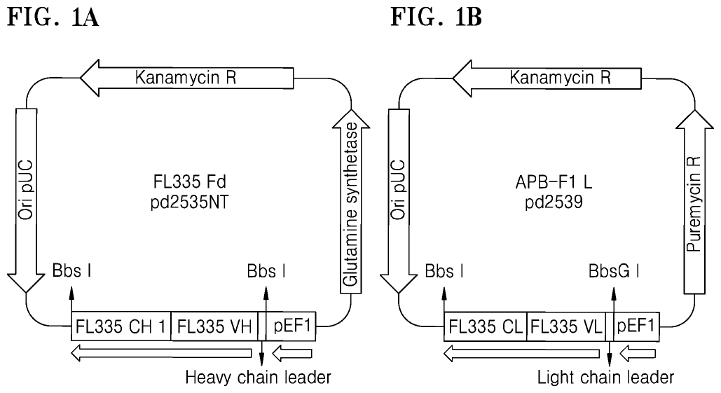

Thereafter, as shown in FIG. 1, the chimeric Fd gene was treated with a restriction enzyme BbsI (Takara, Japan) and inserted into an expression vector pd2535nt (ATUM, Newark, Calif.) to prepare an FL335 Fd pd2535NT vector. The chimeric L gene was treated with restriction enzymes BbsI and BsrGI (Takara, Japan) and inserted into an expression vector pd2539 (ATUM, Newark, Calif.) to prepare an APB-F1 L pd2539 vector.

(2) Preparation of expression vector of feline serum albumin or natural feline granulocyte colony-stimulating factor.

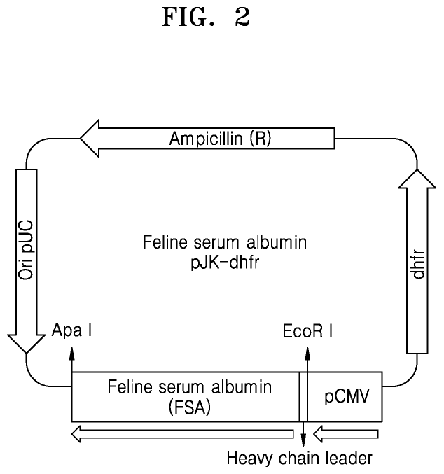

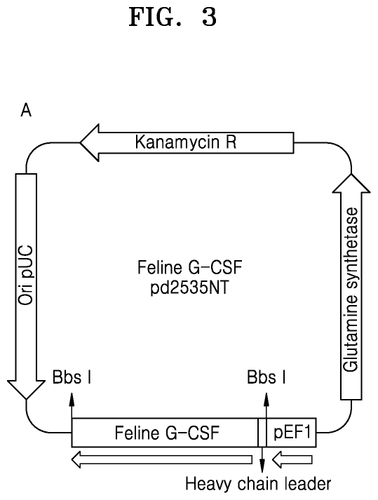

As shown in FIG. 2, feline serum albumin gene was treated with restriction enzymes EcoRI (Takara, Japan) and ApaI (Takara, Japan) and inserted into a pJK expression vector to prepare a feline serum albumin pJK-dhfr vector. Further, as shown in FIG. 3, naturally occurring fGCSF gene was subjected to PCR using primers 1, 9, and 10 under the same conditions as above, and myc tag was linked to the N-terminus of fGCSF, and His tag was linked to the C-terminus thereof. The product was treated with a restriction enzyme Bbs I and then inserted into an expression vector pd2535nt. As a result, a feline GCSF pd2535NT vector was prepared.

(3) Preparation of APB-F1 expression vector.

APB-F(v1) and APB-F(v2), which are two versions of APB-F1, were prepared. In detail, APB-F(v1) was in a form in which naturally occurring (“natural”) GCSF having an 0-sugar chain was linked to FL335, and APB-F(v2) was in a form in which a mutant fGCSF prepared by removing a free cysteine group (C17S) and an O-sugar chain from APB-F(v1) was linked to FL335.

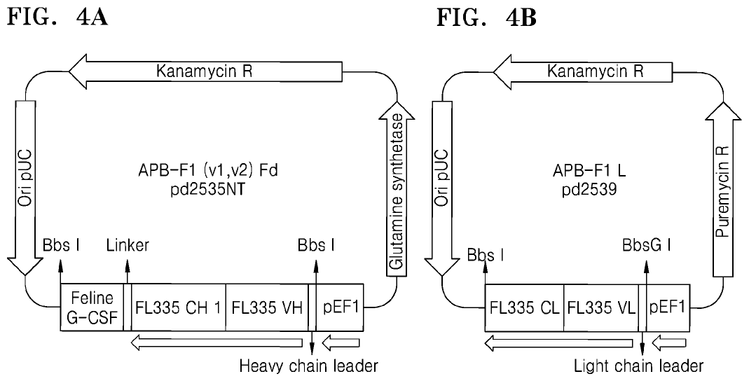

To prepare APB-F(v1) Fd, PCR was carried out using natural fGCSF as a template and primers 14 and IS under the same conditions as above, thereby obtaining a natural fGCSF gene including a linker sequence (GSAGSAPAPAGSGEF; SEQ ID NO:84) in part. PCR was carried out using FL335 Fd gene as a template and primers 1 and 2 to prepare a FL335 Fd gene including the linker sequence in part. Thereafter, linking PCR was carried out using the prepared natural f-GCSF and FL335 Fd gene as a template and primers 1 and 15 under the same conditions as above, thereby preparing APB-F(v1) Fd(FL335 Fd—GSAGSAPAPAGSGEF (SEQ ID NO:84)—natural fGCSF) gene. Then, as shown in FIG. 4A, the prepared gene was treated with a restriction enzyme BbsI, and then inserted into a pd2535nt expression vector to prepare an APB-F1(v1) Fd pd2535NT vector. Meanwhile, as shown in FIG. 4B, the light chain of FL335 inserted in the pd2539 expression vector was used as a light chain of APB-F1.

Further, to prepare APB-F(v2) Fd, His tag was linked to the C-terminus of fGCSF using primers 1, 11, and 12 in the same manner as above, and APB-F(v2) Fd(FL335 Fd—GGGGSGGGGS (SEQ ID NO:85)—mutant fGCSF) gene was synthesized and prepared. In the same manner as above, as shown in FIG. 4A, the prepared gene was treated with a restriction enzyme BbsI, and then inserted into a pd2535nt expression vector to prepare an APB-F1(v2) Fd pd2535NT vector.

Thereafter, to obtain a large amount of the gene for CHO cell transfection, a mixture of DH5 cells (RBC royal bank, Canada) and the gene were heat-shocked at 42° C. for 45 sec, and thus the plasmid DNA was inserted into the cells. Thereafter, the plasmid DNA was obtained using a DNA prep kit (GeneAll, South Korea).

### Preparation Example 2. Construction of Transient Expression System of Recombinant Protein

To express the proteins such as FL335 Fab, feline serum albumin, natural fGCSF, APB-F1(v1), APB-F1 (v2), etc., an ExpiCHO Expression System was used in this experiment. In detail, ExpiCHO-S™ cells (ThermoFisher Scientific) were cultured in Erlenmeyer flasks under conditions of 37° C., 5% CO₂, and 140 rpm using ExpiCHO expression media (Gibco, ThermoFisher Scientific). Thereafter, 10 μl of the cell culture and 10 μl of trypan blue were mixed, and then 10 μl of the mixture was added to cell counting chamber slides. Then, changes in the number of cells and cell viability were measured using a Countess H Automated Cell Counter (Invitrogen) machine. Thereafter, cell transfection using the expression vector prepared in Preparation Example 1 was carried out using an OptiPRO SFM medium (ThermoFisher Scientific) and an ExpiFectamine™ CHO reagent (ExpiFectamine™ CHO Transfection Kit). 18 hr to 22 hr after transfection (day 0), ExpiFectamine™ CHO Enhancer and ExpiCHO™ Feed were added to the culture medium. When the cell density reached 2×10⁶ cells/mL, the transfected cells were cultured after changing the culture conditions to 32° C., 5% CO₂, and 140 rpm. On day 5 after transfection, ExpiCHO™ Feed was further added to the culture medium, and cells were cultured until cell viability reached 80%, and then the culture medium containing the transfected cells was recovered.

Expression patterns and levels of the proteins in the recovered culture medium were examined by Western blotting. First, 1 μg/well of the protein sample was loaded on a 4% to 15% gradient gel, followed by electrophoresis at 150 V for 50 min. Thereafter, the corresponding proteins were transferred onto a nitrocellulose transfer membrane using a transfer buffer (Tris base, glycine, SDS, 20% methanol) at constant 400 mA for 60 min. Then, a 3% skim milk solution at pH 7 was added to the membrane, onto which the proteins had been transferred, and blocked for 1 hr under shaking at room temperature. The membrane was washed with a wash buffer (0.1% tween in 1×PBS) under shaking. Thereafter, detection antibodies were diluted with the 3% skim milk solution at pH 7, and allowed to react with the blocked membrane at room temperature for 1 hr. The detection antibodies used for respective proteins are as follows. For APB-F1(v1, v2) and fGCSF (natural, mutant), rabbit anti-Fe GCSF pAb(1^{st} Ab)(Aprilbio, South Korea) and donkey anti-rabbit IgG(H+L) HRP (2^{nd} Ab)(Jackson Immuno Research, West Grove, Pa.) were used. For FL335 Fab, 1^{st} Ab goat anti-cat light chain Ab (Bethyl Laboratories, Montgomery, Tex.), 2^{nd} Ab goat IgG-Fc fragment cross antibody antibody(Bethyl) or 1^{st} Ab goat anti-Cat F(ab)2-biotin (Jackson Immuno Research, Bar Harbor, Me.), 2^{nd} Streptavidin-HRP (GE Healthcare, Illinois, Chicago) were used. For FSA, anti-His Tag HRP (BioLegend, Sandiego, Calif.) was used. Thereafter, the membrane was washed with a wash buffer, and 1 mL of TMB (Surmodics, Eden prairie, Minnesota) was added thereto to allow a substrate reaction for 1 min to 5 min. Then, the substrate reaction was terminated by adding tertiary distilled water thereto. The mixture of TMB and tertiary distilled water was discarded, and protein expression patterns and levels were examined.

### Preparation Example 3. Preparation of Stable Cell Line for Production of Recombinant Protein

To prepare a stable cell line producing FL335 Fab, natural fGCSF, APB-F1(v1), APB-F1(v2), etc., GS null CHO-K1 cell (HD-BIOP3 cells, Horizon Discovery, UK, Cambridge) was used, and the gene prepared by DNA cloning was used to perform stable transfection as follows. As a basic medium and a production medium, CD FortiCHO™ Media (Life technologies, Carlsbad, Calif.) was used, and subculture was performed using Erlenmeyer flasks (Corning) under conditions of 37° C., 5% CO₂, and 125 rpm. Thereafter, 10 μl of the cell culture and 10 μl of trypan blue were mixed, and then 10 μl of the mixture was added to cell counting chamber slides. Then, changes in the number of cells and cell viability were measured using a Countess II Automated Cell Counter (Invitrogen) machine. For transfection to GS null CHO-K1 cells, two tubes, each containing 600 μl of OptiPRO SFM medium, were prepared, and then 37.5 μg of the plasmid DNA was added to any one of the tubes, and 37.5 μl of Freestyle™ Max reagent (Invitrogen, Thermo Fisher Scientific) was added to the other tube. Then, each of the two tubes was mixed by careful shaking, and incubated at room temperature for 5 min. Thereafter, an SFM medium containing a Freestyle™ Max reagent was transferred to the tube containing the plasmid DNA, and mixed by careful shaking, and allowed to react at room temperature for 20 min to 25 min. Thereafter, the mixture was carefully dispensed to GS null CHO-K1 cells, and cultured under conditions of 37° C., 5% CO₂, and 125 rpm. After 2 days, the culture medium was replaced by CD FortiCHO™ Media without glutamine, and then pool selection was carried out using 50 M L-methionine sulfoximine and 10 μg/mL of puromycin.

After completing the pool selection of the stable cell lines, ELISA was carried out to examine productivity of the recombinant protein in each cell line. Human serum albumin (Sigma Aldrich, Saint Louis, Mo.) was used as a capture antigen (Ag) for APB-F1 (v1, v2), FL335, and Rat anti-Human GCSF Ab (SouthenBiotech, Canada) was used as a capture Ag for fGCSF (natural, mutant). 100 μl of the capture Ag for each sample was added at a concentration of 1 μg/mL to a MaxiSorp NUNC Immuno ELISA plate (Thermo Fisher, Waltham, Mass.), and then incubated at 4° C. overnight to coat the plate with the capture Ag. Thereafter, 300 μl of PBST (0.1% (v/v) Tween (Thermo Fisher)-containing phosphate buffered saline (PBS)) supplemented with 3% bovine serum albumin (BSA) was added to each well, and blocked for 3 hr at room temperature, and each well was washed with 300 μl of a wash buffer (PBST 0.1%). This procedure was repeated three times. 0.3% PBA (in PBST 0.1%) was used as a solution for diluting the sample and antibodies, and 100 μl of the sample diluted with the dilution solution was dispensed to each well, and allowed to react for 1 hr at room temperature. Washing was performed three times in the same manner as above, and 100 μl/well of the detection antibody was dispensed and allowed to react for 1 hr at room temperature. Here, rabbit anti-Fe GCSF pAb (1^{st} Ab) (Aprilbio, KR) and donkey anti-rabbit IgG (H+L) HRP (Jackson Immuno Research, West Grove, Pa.) were used as detection antibodies of APB-F1 (v1, v2), fGCSF, and goat anti-Cat IgG (H+L)-HRP was used as a detection antibody of FL335.

Further, each of the purified proteins was used as a standard, and their productivity was measured. In detail, binding signals were detected by measuring absorbance at 450 nm using an ELISA reader and 3,3′,5,5′-tetramethyl benzidine (TMB) as a substrate.

### Preparation Example 4. Isolation and Purification of Recombinant Protein

The recombinant protein was isolated and purified using an AKTA pure 150 L (GE Healthcare, Chicago Ill.) equipment by the following method.

(1) Isolation and purification of natural or mutant fGCSF.

Natural fGCSF or mutant fGCSF was purified using an immobilized-metal affinity chromatography (IMAC) purification technique and a two-stage ion-exchange chromatography purification technique. First, 300 ml of a culture medium produced by using CD FortiCHO™ Media was centrifuged under refrigerator conditions at 4,000 rpm for 20 min to collect the supernatant, which was then filtered using a 0.2 μm membrane filter to prepare the supernatant. 300 ml of the prepared culture supernatant was applied at a flow rate of 45 cm/h to a 5 ml prepacked Ni-NTA His⋅Bind@ Resin column equilibrated with 10 CVs of 20 mM sodium phosphate at pH 7.4 and 500 mM NaCl buffer, and thus the sample was bound thereto. Then, the column was washed with 20 mM sodium phosphate at pH 7.4, 500 mM NaCl, and 5 mM imidazole buffer to UV 280 nm 10 mAU or less at a flow rate of 60 cm/h. After completely washing the column, 50 mM sodium phosphate at pH 8.0, 300 mM NaCl, and 500 mM imidazole buffer were used as protein elution buffers, and elution was performed at a flow rate of 60 cm/h with concentration gradients of the elution buffer. Thus, elution fractions at respective concentrations were collected. Isolation and purification results were examined by SDS-PAGE analysis, and to perform a subsequent purification, dialysis was performed using a 20 mM sodium citrate buffer at pH 5.5 under refrigerator conditions overnight. fGCSF sample, of which buffer was replaced by 20 mM sodium citrate buffer at pH 5.5, was applied at a flow rate of 60 cm/h to a 5 ml prepacked Hitrap SP HP column equilibrated with 10 CVs of 20 mM sodium citrate buffer at pH 5.5, and thus the sample was bound thereto. Then, the column was washed with 20 mM sodium citrate buffer at pH 5.5 to UV 280 nm 10 mAU or less at a flow rate of 60 cm/h. After completely washing the column, 20 mM sodium citrate at pH 6.5 and 1 M NaCl buffer were used as protein elution buffers, and elution was performed at a flow rate of 60 cm/h with concentration gradients of the elution buffer. Thus, elution fractions of UV 280 nm 10 mAU or more were collected. Thereafter, isolation and purification results were examined by SDS-PAGE analysis, and to perform a subsequent purification, dialysis was performed using a 20 mM sodium citrate buffer at pH 5.5 under refrigerator conditions overnight. fGCSF sample, of which buffer was replaced by 20 mM sodium citrate buffer at pH 5.5, was applied at a flow rate of 60 cm/h to a 5 ml prepacked POROS XQ column equilibrated with 10 CVs of 20 mM sodium citrate buffer at pH 5.5, and thus the sample which was not bound to the column but passed though the column was collected. Thereafter, isolation and purification results were examined by SDS-PAGE analysis, and impurities were removed using a 0.2 μm filter, and a UV protein quantitation method was used to quantify the protein, which was then stored at −20° C. until use.

(2) Isolation and purification of APB-F1.

APB-F1(v1, v2) was purified by an affinity chromatography using Capto L resin and a two-stage ion-exchange chromatography purification technique. First, 900 ml of APB-F1 (v1, v2) culture medium produced by using CD FortiCHO™ Media was centrifuged under refrigerator conditions at 4,000 rpm for 20 min to collect the supernatant, which was then filtered using a 0.2 μm membrane filter to prepare the supernatant. 900 ml of the prepared culture supernatant was applied at a flow rate of 120 cm/h to a 34 ml prepacked Capto L column equilibrated with 10 CVs of 1×PBS, and thus the sample was bound thereto. Then, the column was washed with 20 mM sodium citrate at pH 6.0 and 1% D-mannitol buffer to UV 280 nm 10 mAU or less at a flow rate of 120 cm/h. After completely washing the column, 50 mM sodium citrate at pH 3.0 and 3% D-mannitol buffer were used as protein elution buffers, and 100% of the elution buffer was applied at a flow rate of 120 cm/h to collect protein elution fractions of UV 280 nm 10 mAU or more. 1 M Tris-Cl solution at pH 8.0 was added to the collected elution fraction, and pH was slightly acidified to 6.0. Then, impurities were removed using a 0.2 μm filter. The sample obtained by AC purification was applied at a flow rate of 153 cm/h to a 10 ml CM sepharose FF column equilibrated with 10 CVs of 20 mM sodium citrate buffer at pH 6.0, and thus the sample was bound thereto. Then, the column was washed with 20 mM sodium citrate buffer at pH 6.0 to UV 280 nm 10 mAU or less at a flow rate of 153 cm/h. After completely washing the column, 20 mM sodium citrate at pH 6.0 and 1 M NaCl buffer were used as protein elution buffers, and elution was performed at a flow rate of 153 cm/h with concentration gradients of the elution buffer. Thus, elution fractions of UV 280 nm 10 mAU or more were collected. Isolation and purification results were examined by SDS-PAGE analysis, and to perform a subsequent purification, dialysis was performed using a 20 mM sodium citrate buffer at pH 6.0 under refrigerator conditions overnight. APB-F1(v1, v2) samples, of which buffer was replaced by 20 mM sodium citrate buffer at pH 6.0, were applied at a flow rate of 34 cm/h to a 35 mil prepacked POROS 50HQ equilibrated with 10 CVs of 20 mM sodium citrate buffer at pH 6.0, and thus the sample which was not bound to the column but passed though the column was collected. Thereafter, isolation and purification results were examined by SDS-PAGE analysis, and impurities were removed using a 0.2 μm filter.

### Preparation Example 5. SDS-PAGE Analysis

The protein sample was treated under three kinds of conditions: \textcircled{1} reducing conditions (10% glycerol, 1% lithium dodecyl sulfate (LDL), 0.2 M triethanolamine-Cl at pH 7.6, 1% Ficoll®-400, 0.000625% phenol red, 0.000625% coomassie G250, 0.5 mM EDTA disodium, 1.25% 2-Mercaptoethanol, 10 min-boiling), \textcircled{2} non-reducing conditions (10% glycerol, 1% lithium dodecyl sulfate(LDL), 0.2 M triethanolamine-Cl at pH 7.6, 1% Ficoll®-400, 0.000625% phenol red, 0.000625% coomassie G250, 0.5 mM EDTA disodium, 10-min boiling), and \textcircled{3} non-reducing conditions (not boiling) (10% glycerol, 0.375 M triethanolamine-CI at pH 6.8, 0.005% Bromophenol blue, not boiling). Thereafter, the protein sample was loaded at a concentration of 1 μg/well on a 4%-15% gradient precast gel, followed by electrophoresis under conditions of constant 150 V and 50 min. After electrophoresis, the separated gel was stained with an Ez-Gel staining solution for 1 hr, and then destained with water. Thereafter, protein analysis was performed by comparing with an Excelband™ Enhanced 3-color high range protein marker.

### Preparation Example 6. Size-Exclusion Chromatography

To examine purity of APB-F1(v1, v2), SE-HPLC was performed using an Agilent 1260 Infinity II system. A 7.8×300 mm TSK gel UltraSW aggregate (Tosoh Biosciences, Japan) column was used as an SE-HPLC column, and in detail, SE-HPLC was performed under conditions of \textcircled{1} Column temperature: 20° C., \textcircled{2} mobile phase A: 20 mM sodium citrate pH 5.5, 100 mM NaCl, \textcircled{3} flow rate: 0.7 ml/min, \textcircled{4} Wavelength: 280 nm, \textcircled{5} injection: 40 μg, \textcircled{6} gradient: isocratic.

### Example 1. Expression and Production of FL33S and Feline Serum Albumin

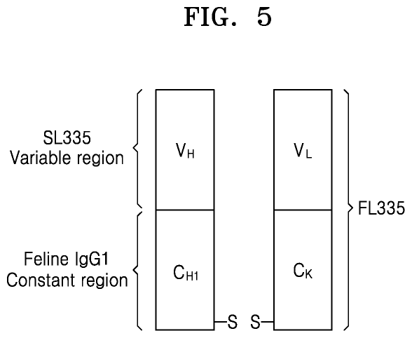

In this Example, expression and production of FL335 antibody fragment and feline serum albumin according to Preparation Example were examined. FL335 comprises human V_{H} and V_{L} sequences of SL335 and feline IgG1 CH1 (delta C) and CL-kappa, as shown in FIG. 5. To prepare FL335, SL335 V_{H} and feline IgG1 C_{H1} were linked to each other by linking PCR to prepare FL335 Fd gene, and in the same manner, SL335 V_{L} and feline CL-kappa were linked to each other to prepare FL335 L gene. Thereafter, FL335 Fd, FL335 L, and feline serum albumin produced and purified according to Preparation Example were identified by SDS-PAGE.

FL335 Fd or FL335 L was inserted into an expression vector, respectively and then protein expression by the expression vector was identified by transient expression using an ExpiCHO expression system. Thereafter, a culture medium obtained by flask production of the stable cell line was subjected to purification procedures including affinity chromatography and two-stage ion-exchange chromatography, and proteins purified from the supernatant of GS null CHO-K1 cell culture medium were identified by SDS-PAGE on a 4%-15% gradient gel.

Feline serum albumin was also directly prepared by gene synthesis, and for isolation and purification and analysis, myc tag was linked to the N-terminus and his tag was linked to the C-terminus, and the product was inserted into an expression vector pJK plasmid. Then, protein expression by the expression vector was identified by transient expression using an ExpiCHO expression system. Further, a culture medium obtained by flask production of the stable cell line was subjected to isolation and purification, and proteins purified therefrom were identified by SDS-PAGE.

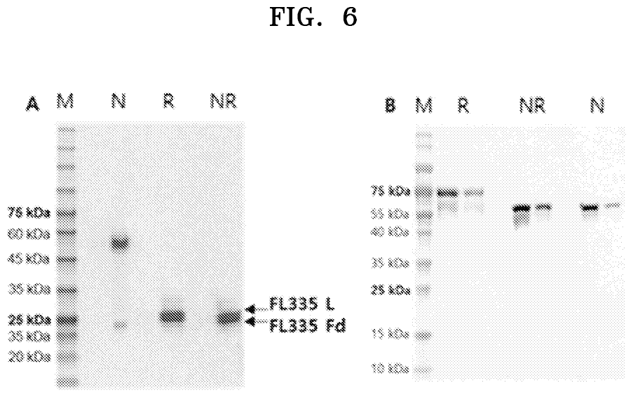

FIGS. 6A and 6B show identification of FL335 Fd, L and feline serum albumin in the culture media after expression and purification processes according to one exemplary embodiment, wherein FIG. 6A shows the SDS-PAGE results of identifying FL335, and FIG. 6B shows the SDS-PAGE results of identifying feline serum albumin.

As shown in FIG. 6A, protein bands corresponding to FL335 Fd having a theoretical molecular weight of 23.492 kDa and FL335 L having a theoretical molecular weight of23.877 kDa were identified, and a protein band corresponding to FL335 Fab having a theoretical molecular weight of 47.352 kDa was also identified. As shown in FIG. 6B, a protein band corresponding to feline serum albumin having a theoretical molecular weight of 67.8 kDa was also identified.

### Example 2. Examination of Binding Ability of FL335 to Feline Serum Albumin

In this Example, binding ability of FL335 to feline serum albumin was examined. In detail, feline serum albumin was diluted with a carbonate coating buffer at pH 9.6 at a concentration of 1 μg/mL, and 100 μl of the dilution was added to each well of a MaxiSorp NUNC Immuno ELISA plate, and left at 4° C. overnight to coat the plate with feline serum albumin. PBST (0.1% (v/v) Tween-containing PBS) supplemented with 3% bovine serum albumin was used as a blocking buffer, and 300 μl of the blocking buffer was added to each well to perform blocking at room temperature. Thereafter, washing was performed by dispensing 300 μl of a wash buffer (PBST) to each well, and this procedure was repeated three times. 0.3% PBA (in PBST 0.1%) was used as a solution for diluting the antibody fragment or sample, and 100 μl of the dilution was dispensed to the feline serum albumin-coated well, and allowed to react at room temperature for 1 hr. Washing was performed three times in the same manner as above. Thereafter, 1:3000 dilution of goat anti-human kappa-HRP as a SL335 detection 1^{st} antibody was added to the well, and 1:3000 dilution of goat anti-cat light chain Ab as a FL335 detection 1^{st} antibody and 1:5000 dilution of goat IgG-Fc fragment cross antibody as a FL335 detection 2^{nd} antibody were added to the well. An antigen-antibody reaction thereby was allowed at room temperature for 1 hr, and then binding signals were detected by measuring absorbance at 450 nm using an ELISA reader and 3,3′,5,5′-TMB as a substrate. Meanwhile, in this Example, to examine functionality of the FL335 antibody fragment binding to albumin, its binding with human serum albumin or feline serum albumin was compared with that of SL335 which is a Fab fragment specifically binding to human albumin.

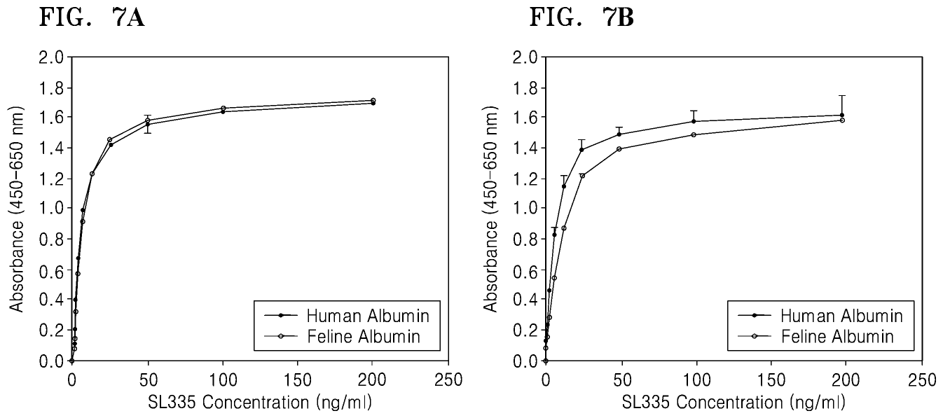

FIGS. 7A and 7B show binding ability of FL335 to serum albumin, wherein FIG. 7A shows ELISA results of identifying the binding ability of SL335 to human serum albumin and feline serum albumin, and FIG. 7B shows ELISA results of identifying the binding ability of FL335 to human serum albumin and feline serum albumin.

As shown in FIG. 7, the FL335 antibody fragment showed binding ability to all the serum albumins in a similar level to that of SL335. These experimental results indicate that even when the feline heavy chain constant 1 domain or light chain constant domain (CH1, Cκ) is bound to the heavy chain or light chain variable region domain of the existing antibody specifically binding to anti-serum albumin, i.e., SL335, the binding ability to albumin is maintained.

### Example 3. Expression and Production of Feline Granulocyte Colony-Stimulating Factor

In this Example, expression and production of fGCSF protein according to Preparation Example were examined. For prevention of miss folding that can occur during the protein production process, and for convenience in purification and processing processes, two kinds of fGCSFs, i.e., natural fGCSF and mutant fGCSF were prepared. In detail, mutant fGCSF was prepared by C17S substitution to remove free cysteine from natural fGCSF and by T133A substitution to remove an O-sugar chain from natural fGCSF. To prepare cell lines each stably expressing natural fGCSF or mutant fGCSF, GS null CHO-K1 cells were transfected therewith, and selected using L-methionine sulfoximine and puromycin over about 4 weeks to prepare stable cell lines. Thereafter, a culture medium obtained by flask production of the stable cell line was subjected to purification procedures including immobilized-metal affinity chromatography and two-stage ion-exchange chromatography, and proteins purified from the supernatant of GS null CHO-K1 cell culture medium were identified by SDS-PAGE on a 4%-15% gradient gel.

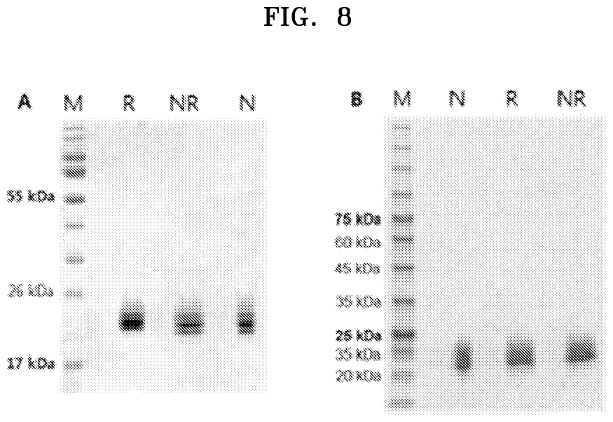

FIGS. 8A and 8B show identification of natural fGCSF and mutant fGCSF in the culture media after expression and purification processes according to one exemplary embodiment, wherein FIG. 8A shows the SDS-PAGE results of identifying natural fGCSF, and FIG. 8B shows the SDS-PAGE results of identifying mutant fGCSF.

As shown in FIG. 8, protein bands each corresponding to natural fGCSF and mutant fGCSF were identified, and the size of the protein band of mutant fGCSF was reduced due to removal of free cysteine and O-sugar chain under non-reducing (not boiled) conditions, as compared with that of the natural fGCSF.

### Example 4. Expression and Production of APB-F1

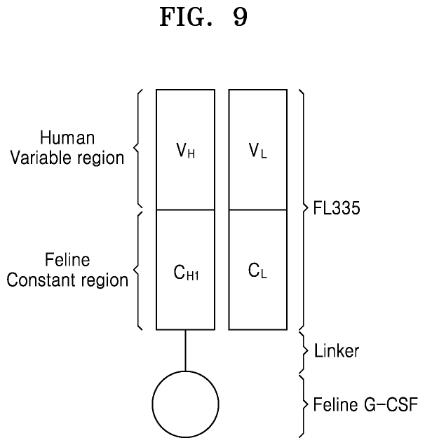

In this Example, expression and production of APB-F1 according to Preparation Example were examined. As shown in FIG. 9, APB-F1 was prepared as two kinds of proteins according to fGCSF. In other words, APB-F1(v1) is a protein obtained by linking natural fGCSF to the C-terminus of FL335 Fd, and APB-F1(v2) is a protein obtained by linking mutant fGCSF to the C-terminus of FL335 Fd. To prepare cell lines each stably expressing APB-F1(v1) or APB-F1(v2), GS null CHO-K1 cells were transfected therewith, and selected using L-methionine sulfoximine and puromycin over about 4 weeks to prepare stable cell lines. Thereafter, a culture medium obtained by flask production of the stable cell line was subjected to purification procedures including affinity chromatography and two-stage ion-exchange chromatography, and proteins purified from the supernatant of GS null CHO-K1 cell culture medium were identified by SDS-PAGE on a 4%-15% gradient gel.

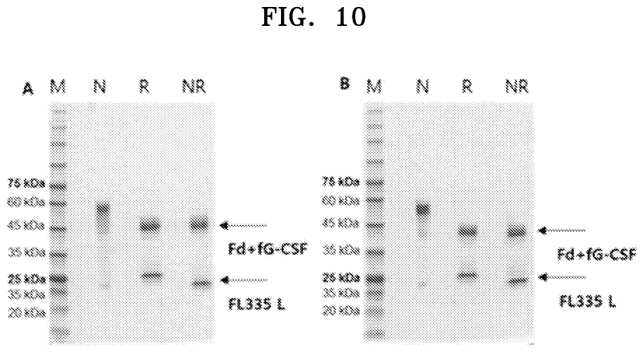

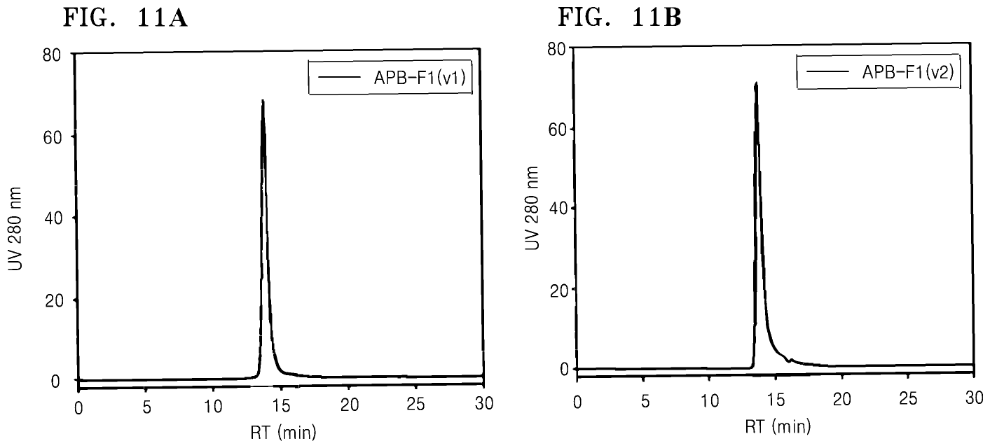

FIG. 10A and 10B show identification of APB-F1 in culture media after expression and purification processes according to one exemplary embodiment, wherein FIG. 10A shows the SDS-PAGE results of identifying APB-F1(v1), and FIG. 10B shows the SDS-PAGE results of identifying APB-F1(v2). Further, FIGS. 11A and 11B show purity of APB-F1 samples after expression and purification processes according to one exemplary embodiment, wherein FIG. 11A shows the SEC-HPLC results of analyzing the APB-F1(v1) sample, and FIG. 11B shows the SEC-HPLC results of analyzing the APB-F1(v2) sample.

As shown in FIG. 10, protein bands each corresponding to APB-F1(v1) or APB-F1(v2) were identified, and as in the above results, the size of the protein band of APB-F1(v2) including mutant fGCSF was reduced due to removal of free cysteine and O-sugar chain under non-reducing (not boiled) conditions, as compared with that of APB-F1(v1) including natural fGCSF. Further, as shown in FIG. 11, APB-F1(v1) and APB-F1(v2) samples obtained through a series of preparation and purification processes described above were confirmed to have a high purity of about 98%.

### Example 5. Evaluation of Biological Activity of APB-F1

In this Example, biological activity of APB-F1 was evaluated by measuring EC₅₀ of APB-F1. In detail, M-NFS60 cells (ATCC No. 1838) were subjected to a cell proliferation assay. FL335 was used as a negative control group, and human granulocyte colony-stimulating factor (hGCSF I.S, rDNA Derived, 2nd International Standard, NIBSC code: 09/136) which is a biologically active standard decided by WHO was used as a positive control group, and Filgrastim, PEG-Filgrastim, and fGCSF were used as comparative groups. First, M-NFS60 cells were cultured in an RPMI-1640 medium supplemented with 1% FBS, and maintained under culture conditions of 37° C. and 5% CO₂. Thereafter, the cells were diluted at a density of 6×10⁴ cells/mi with RPMI-1640 (Gibco) medium, and 100 μl of the dilution was added to a 96-well plate. Subsequently, APB-F1, a control material, and a control material were diluted at a concentration of 0.01 pM to 1000 pM with RPMI-1640 medium, and 100 μl of the dilution was added to each well in triplicate, followed by incubation under conditions of 37° C. and 5% C02 for 42 hr. Thereafter, a cell counting kit-8 solution was added at a concentration of 20 μl/well to the cultured cells, and then allowed to react for 6 hr. To examine cell viability, absorbance at 450 nm was measured using a microplate reader.

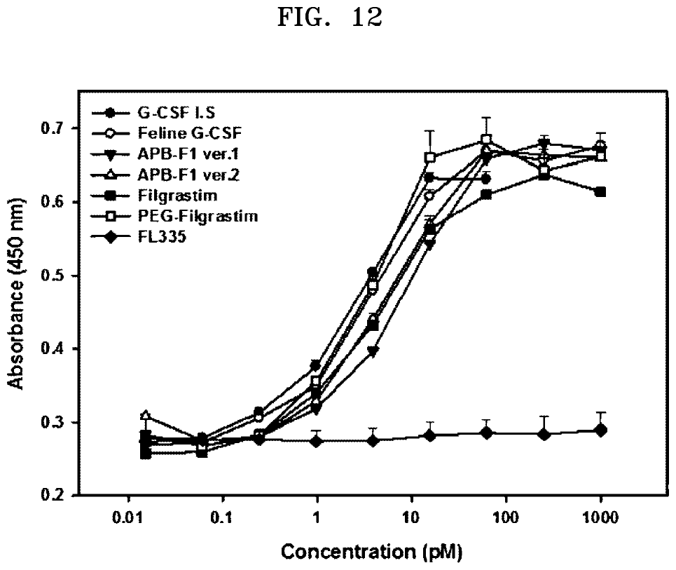

FIG. 12 shows results of a proliferation assay for M-NFS60 cells, performed by using APB-F1. As shown in FIG. 12, it was confirmed that hGCSF I.S, fGCSF, APB-F1(v1), and APB-F1(v2) showed EC₅₀ of 2.45 pM, 3.59 pM, 8.32 pM, and 5.67 pM, respectively, and Filgrastim and PEG-Filgrastim showed EC₅₀ of 3.94 pM and 3.07 pM, respectively. In detail, activity of Filgrastim measured in this experiment was 0.6×10⁸ U/mg, within the range of (0.6±1.0)×10⁸ U/mg which is the known activity of Filgrastim, indicating effectiveness of the present experiment. Further, activity of fGCSF and PEG-Filgrastim was measured as 6.5×10⁶ U/mg and 3.6×10⁷ U/mg, respectively, and activity of APB-F1(v1) and APB-F1(v2) was measured as 7.8×10⁶ U/mg and (8.7±0.3)×10⁶ U/mg, respectively, indicating similar levels of the activity. These results indicate that APB-F1(v2) maintained its biological activity despite removal of free cysteine and O-sugar chain from fGCSF, and APB-F1(v2) was chosen as the APB-F1 protein in the following Examples for convenience of production, isolation, and purification processes of the recombinant protein.

### Example 6. Intact Mass Spectrometry

In this Example, intact mass spectrometry was performed under reducing conditions to examine an accurate molecular weight of APB-F1. In this experiment, Dionex UHPLC and Q-TOF5600+MS/MS system were used. Mass of APB-F1 was measured using a 1.7-μm Acquity UPLC® BEH130 C4 column, and acetonitrile as a mobile phase at a flow rate of 0.3 ml/min.

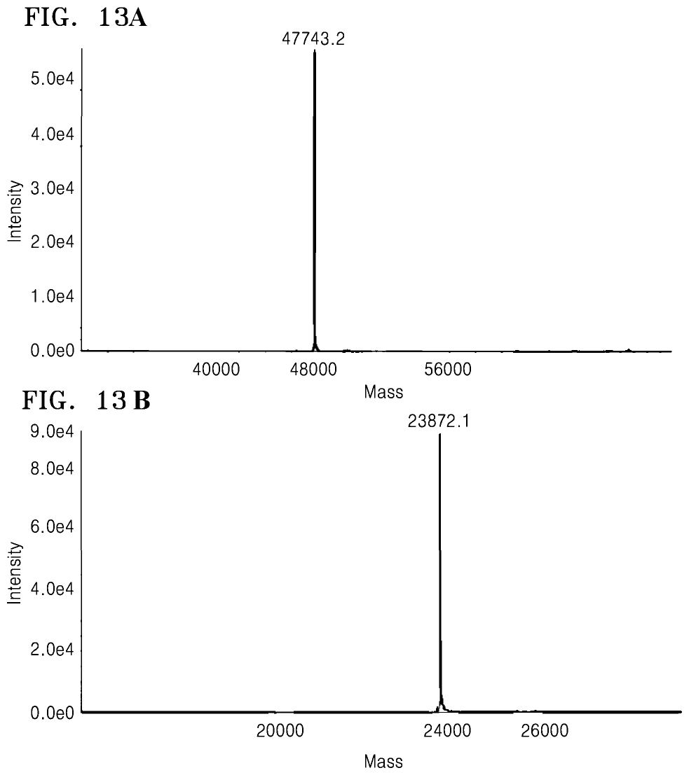

FIGS. 13A and 13B show the molecular weight of APB-F1, examined by intact mass spectrometry, wherein FIG. 13A shows the results of examining the mass of APB-F1 Fd and FIG. 13B shows the results of examining the mass of APB-F1 L. As shown in FIG. 13, the mass of APB-F1 Fd was measured as 47.743 kDa, and the mass of APB-F1 Fd L was measured as 23.872 kDa.

### Example 7. N-Terminal Sequencing

In this Example, to identify the specific N-terminal sequence of APB-F1 and to examine accuracy of the process such as removal of the signal sequence after expression of the recombinant protein, N-terminal sequencing was performed. This experiment was assigned to and performed at ProteomeTech Inc., and an Edman degradation method and LC-MS/MS were used. The Edman degradation method was performed using a protein sequencer for an exposure time of 3.5 min to 18.0 min under a detector scale condition of 0.005 AUFS, based on relative retention time (dptu) of a reference material (PTH-AA, 8.0 pM), to analyze the N-terminal sequence of APB-F1 (10.0 pM). Table 2 shows the results of analyzing the N-terminal sequence of APB-F1 by the Edman degradation method.

TABLE 2

| | | APB-F1(v2) Fd chain | APB-F1(v2) Light chain |
|---|---|---|---|
| Cycle | Residue | Amino Acid | Amino Acid |
| 1 | Blank | — | — |
| 2 | Standard | — | — |
| 3 | 1 | — | Asp(D) |
| 4 | 2 | — | Ile(I) |
| 5 | 3 | — | Val(V) |
| 6 | 4 | — | Leu(L) |
| 7 | 5 | — | Thr(T) |

As shown in Table 2, the N-terminal sequence of APB-F1 was identified as DIVLT. In contrast, it was impossible to identify the sequence of APB-F1 Fd due to blocking of the reaction resulting from the biological conversion of the first residue glutamine (Gin) to polyglutamate (pGlu).

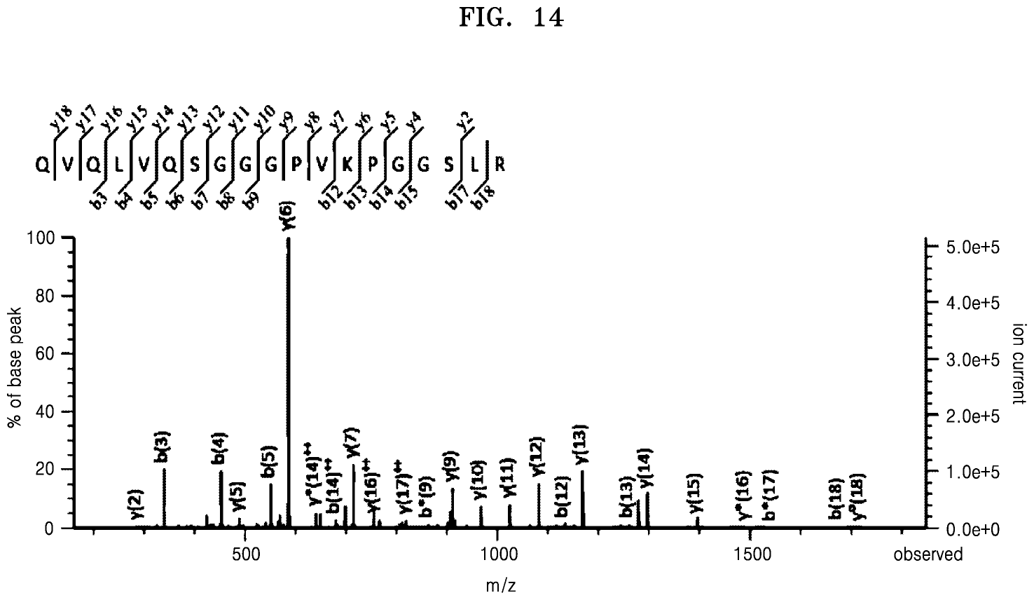

Then, LC-MS/MS was performed using a NanoUPLC, LTQ-orbitrap-mass spectrometer under conditions of Peptide Mass Tolerance (±10 ppm) Fragment Mass Tolerance (±0.8 Da), Max Missed Cleavages(2) within the range of 300 m/z to 2,000 m/z to analyze nucleotide sequences of QVQLVQSGGGPVKPGGSLRLSCAAS (N-terminal of SEQ ID NOS:22 and 23). Analysis was performed using Proteome Discoverer, MASCOT software. Table 3 shows the LC-MS/MS results of analyzing the N-terminal sequence of APB-F1 Fd using Proteome Discoverer software, and FIG. 14 shows the LC-MS/MS results of analyzing the N-terminal sequence of APB-F1 Fd using MASCOT software.

TABLE 3

| | | | | | | | | | | Peptide (N- | |
|---|---|---|---|---|---|---|---|---|---|---|---|
| | | | | | | | | | | terminal of | |
| | | | | | | | | | | SEQ ID NOS: | |
| Query | Start | — | End | Observed | Mr (expt) | Mr (calc) | Delta | Score | Expect | 22 and 23) | Modification |
| 348 | 1 | — | 19 | 616.3434 | 1846.0085 | 1846.0061 | 1.28 | 45 | 3.10E−05 | -QVQLVQSGGG | Gln->pyro- |
| | | | | | | | | | | PVKPGGSLR.L | Glu (N-term |
| | | | | | | | | | | | Q) |
| 349 | 1 | | 19 | 924.012 | 1846.0095 | 1846.0061 | 1.82 | 61 | 8.80E−07 | -QVQLVQSGGG | Gln->pyro- |
| | | | | | | | | | | PVKPGGSLR.L | Glu (N-term |
| | | | | | | | | | | | Q) |
| 350 | 1 | — | 19 | 924.0126 | 1846.0107 | 1846.0061 | 2.48 | 78 | 1.70E−08 | -.QVQLVQSGGG | Gln->pyro- |
| | | | | | | | | | | PVKPGGSLR.L | Glu (N-term |
| | | | | | | | | | | | Q) |
| 351 | 1 | — | 19 | 924.0139 | 1846.0133 | 1846.0061 | 3.88 | 48 | 1 80E−05 | -.QVQLVQSGGG | Gln->pyro- |
| | | | | | | | | | | PVKPGGSLR.L | Glu (N-term |
| | | | | | | | | | | | Q) |
| 352 | 1 | | 19 | 924.0154 | 1846.0162 | 1846.0061 | 5.46 | 43 | 5.20E−05 | -QVQLVQSGGG | Gln->pyro- |
| | | | | | | | | | | PVKPGGSLR.L | Glu (N-term |
| | | | | | | | | | | | Q) |
| 353 | 1 | — | 19 | 924.506 | 1846.9974 | 1846.9901 | 3.93 | 73 | 4.70E−08 | -.QVQLVQSGGG | Deamidated |
| | | | | | | | | | | PVKPGGSLR.L | (NQ); |
| | | | | | | | | | | | Gln->pyro- |
| | | | | | | | | | | | Glu (N-term |
| | | | | | | | | | | | Q) |
| 364 | 1 | — | 19 | 622.0192 | 1863.0357 | 1863.0327 | 1.62 | 16 | 0.027 | -.QVQLVQSGGG | |
| | | | | | | | | | | PVKPGGSLR.L | |
| 365 | 1 | | 19 | 622.0197 | 1863.0373 | 1863.0327 | 2.49 | 39 | 0.00014 | -.QVQLVQSGGG | |
| | | | | | | | | | | PVKPGGSLR.L | |
| 366 | 1 | — | 19 | 622.346 | 1864.0162 | 1864.0167 | −0.26 | 34 | 0.00036 | -.QVQLVQSGGG | Deamidated |
| | | | | | | | | | | PVKPGGSLR.L | (NQ) |

As shown in Table 3 and FIG. 14, the N-terminal sequence of APB-F1 Fd (SEQ ID NOS:22 and 23) was identified as Q(pyro-glu)VQLV.

### Example 8. Pharmacokinetic Evaluation of APB-F1

In this Example, pharmacokinetic evaluation was performed to examine absorption, distribution, in-vivo change, and excretion of APB-F1. In detail, a test material of APB-F1 was subcutaneously injected into healthy cats, and blood samples were collected and analyzed. Each group consisted of a total of four cats including two male cats and two non-pregnant female cats. 2 days or 4 days before administration of the test material, immediately after administration of the test material, 2 hr, 6 hr, or 12 hr after administration of the test material, 3 ml of whole blood was collected each time through the jugular vein for a total of 15 times according to the passage of time after the administration of the test material. Immediately after collection, the whole blood was centrifuged for 5 minutes at 3,000 rpm to separate the plasma, which was then stored in a deep freezer (about −70° C.) until use. In this experiment, a group administered with 100 μg/kg of mutant fGCSF protein and a group administered with 360 μg/kg of APB-F1 were set as experimental groups.

Then, the obtained samples were analyzed by ELISA. First, 100 μl of a rat anti-human GCSF Ab solution diluted at a concentration of 1 μg/mL with a carbonate coating buffer at pH 9.6 was loaded on each well of an ELISA plate in an amount of 100 ng/well, and the plate was coated with the antibody by incubation at 2° C. to 8° C. overnight. After removing the solution from the plate, washing was performed once by adding 300 μl of a wash buffer to each well. Thereafter, blocking was performed at room temperature for 3 hr by adding 300 μl of a blocking buffer to each well. After removing the solution from the plate, the plate was turned upside down, and left for 1 hr at room temperature to remove all the remaining solution. Each 100 μl of a standard material and a diluted test solution were added to each well in triplicate. Thereafter, the plate was covered with a sealer, and allowed to react at room temperature and 450 rpm for 90 min, and the plate was washed with the wash buffer four times in the same manner as above. Thereafter, 100 μl of 1:1,000 dilution of rabbit anti-fGCSF pAb which is a primary antibody was added to each well. The plate was covered with the sealer, and allowed to react at room temperature for 1 hr. Subsequently, peroxidase-conjugated AffiniPure Goat anti-Rabbit IgG (H+L) which is a secondary antibody was added to induce an antigen-antibody reaction. In detail, washing was performed in the same manner as above four times, and then 100 μl of 1:10,000 dilution of the secondary antibody was added to each well, and allowed to react at room temperature in the dark for 1 hr. Then, washing was performed in the same manner as above five times, and 100 μl/well of BioFX® TMB Super Sensitive One Component HRP Microwell Substrate was added and allowed to react at room temperature for 5 min to 10 min. Thereafter, 1 N of HCL was added to terminate the substrate reaction. Subsequently, absorbance was measured using a SPECTROstar Nano Microplate reader at a measurement wavelength of 450 nm and a reference wavelength of 650 nm, and statistical analysis was performed using a Phoenix WinNonlin software.

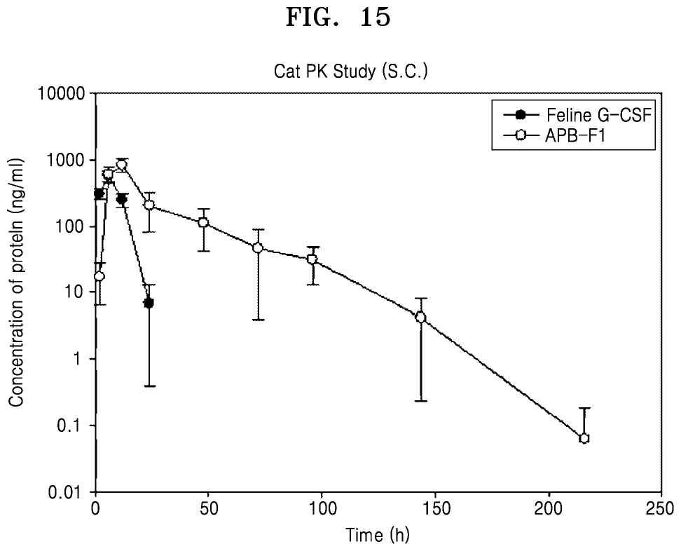

Table 4 and FIG. 15 show the results of pharmacokinetic evaluation of APB-F1 in cats. As shown in Table 4 and FIG. 15, the time taken to reach the maximum blood concentration (Tₘₐₓ) of fGCSF was 6 hr, whereas Tₘₐₓ of APB-F1 was 12 hr, and AUClast of APB-F1 was 19025.4 h-ng/mL, which is an about 3.1-fold increase, as compared with that of fGCSF of 6050.03 h·ng/mL. Further, the maximum blood concentration (Cₘₐₓ) of fGCSF was 576.3 ng/mL, whereas Cₘₐₓ of APB-F1 was 823.9 ng/mL, which is a 1.4-fold increase, as compared with that of fGCSF. The elimination half-life (T½) of APB-F1 was 13.3 hr, which is an about 4.9-fold increase, as compared with that of fGCSF of 2.7 hr. These experimental results indicate that APB-F1 according to one exemplary embodiment has improved pharmacokinetic properties, as compared with mutant fGCSF protein.

TABLE 4

| Test | Dose | t½ (N = 4) | Tₘₐₓ (N = 4) | Cₘₐₓ (N = 4) | CL (N = 4) | AUClast (N = 4) |
|---|---|---|---|---|---|---|
| article | (μg/kg) | h | H | ng/ml | mL/hr/kg | hr*ng/mL |
| fGCSF | 100 | 2.7127844 | 6 | 576.2821 | 16.17919 | 6050.035536 |
| APB-F1 | 360 | 13.280243 | 12 | 823.9247 | 18.9209 | 19025.3947 |

### Example 9. Pharmacodynamic Evaluation of APB-F1

In this Example, pharmacodynamic evaluation was performed to examine physiological and physiochemical actions and effects of APB-F1. In detail, a test material of APB-F1, etc. was subcutaneously injected into healthy cats, and white blood cell levels in blood were measured and analyzed. Each group consisted of a total of four cats including two male cats and two non-pregnant female cats. 3 ml of whole blood was collected each time through the jugular vein according to the passage of time from the administration day (Day 0). Subsequently, the obtained samples were subjected to hematological tests using an automated blood analyzer, and blood biochemical tests using a blood biochemistry analyzer and an automated electrolyte analyzer. In this experiment, 36 μg/kg of APB-F1-administered group, 360 μg/kg of APB-F1-administered group, 10 μg/kg of fGCSF-administered group, 10 μg/kg of Filgrastim (Grasin)-administered group, 100 μg/kg of PEG-filgrastim (Neulasta)-administered group, and 26 μg/kg of FL335 (Fab)-administered group were set as experimental groups, and a vehicle-administered group was set as a control group.

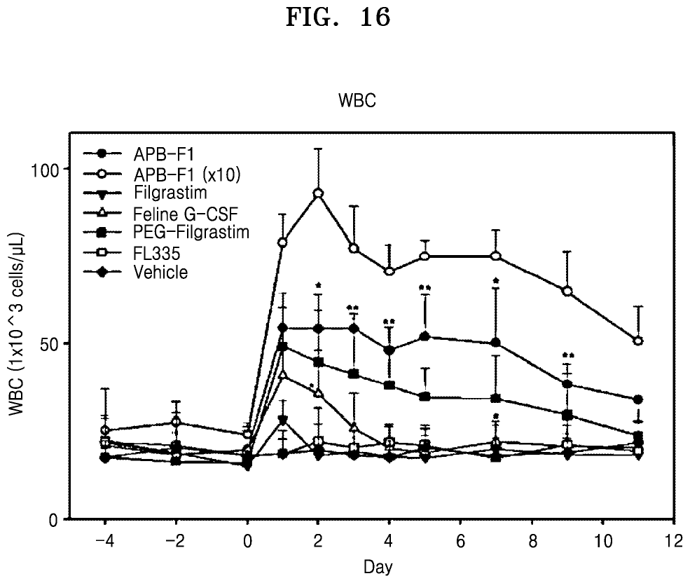

FIG. 16 shows the results of pharmacodynamic evaluation of APB-F1 in cats, wherein white blood cell levels in blood were examined. As shown in FIG. 16, the APB-F1-administered group showed a significantly high level of white blood cells from Day 1 to Day 11 after administration, as compared with the level before administration, and also showed the higher level than the normal range of the level (4.9×10³ cells/μl to 2.0×10⁴ cells/μl) on Day 20 after administration (not shown). Further, the fGCSF-administered group showed a significant difference in the level of white blood cells between day 1 after administration and before administration, but the level was decreased to the normal range of the level on day 5 after administration. The Filgrastim-administered group also showed a significant increase in the level of white blood cells on day 1 after administration, but the level was decreased to the normal range of the level on day 2 after administration. The PEG-Filgrastim-administered group showed a significant high level of white blood cells until day 7 after administration, as compared with that before administration, and the level was maintained at a higher level than the normal range of the level until day 10 after administration.

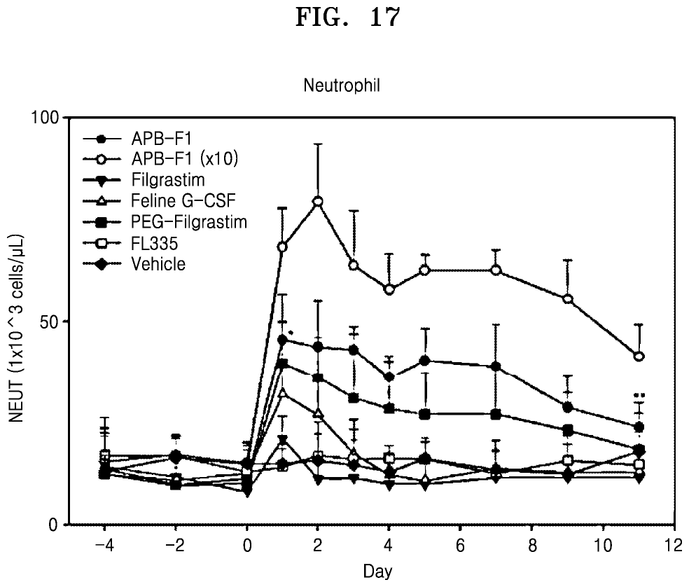

FIG. 17 shows results of examining neutrophil levels in blood. As shown in FIG. 17, the fGCSF or APB-F1-administered group showed a significantly high level of neutrophils in blood on day 1 after administration, as compared with that before administration, but the APB-F1-administered group showed a much higher level of neutrophils in blood than the fGCSF-administered group. Further, the APB-F1-administered group maintained a significantly high level of neutrophils in blood from day 1 to day 11. Similarly, it was observed that the PEG-Filgrastim-administered group maintained a much high level of neutrophils in blood for a long period of time, as compared with the Filgrastim-administered group. Meanwhile, no significant difference was observed between other administered groups and control groups.

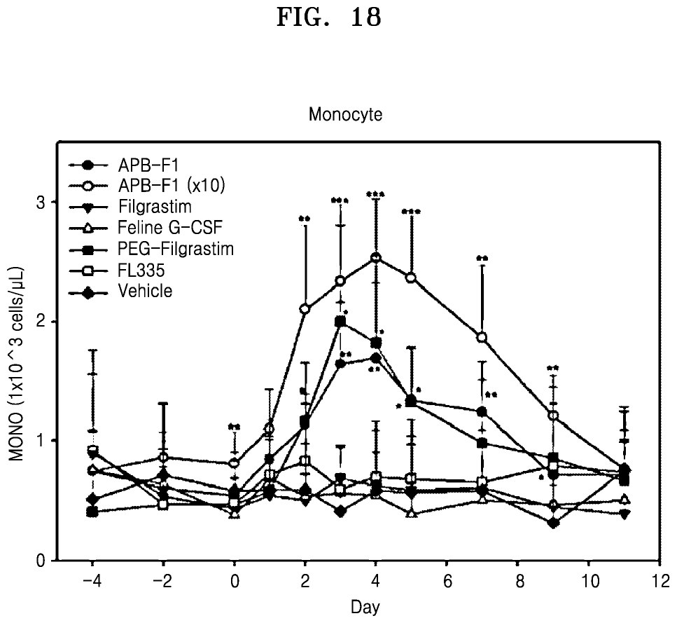

FIG. 18 shows results of examining monocyte levels in blood. As shown in FIG. 18, the APB-F1-administered group showed a significantly high level of monocytes in blood from day 2 to day 9 after administration, as compared with that before administration, and the PEG-Filgrastim-administered group showed a high level of monocytes in blood from day 2 to day 5 after administration, as compared with that before administration. Meanwhile, no significant difference was observed between other administered groups and control groups.

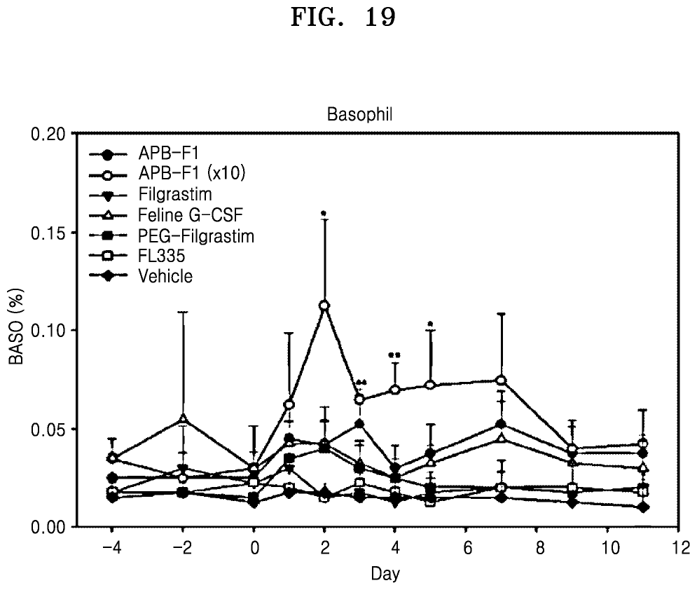

FIG. 19 shows results of examining basophil levels in blood. As shown in FIG. 19, the APB-F1-administered group showed a significantly high level of basophils in blood from day 2 to day 7 after administration, as compared with the normal range of the level.

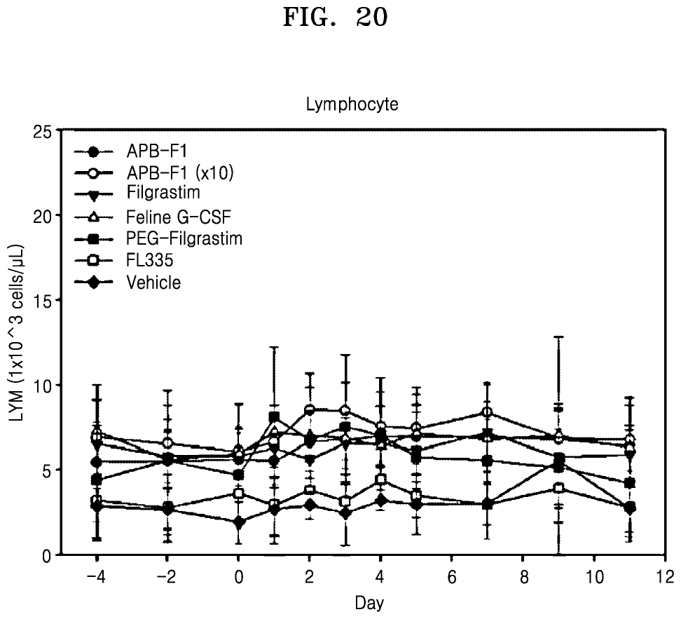

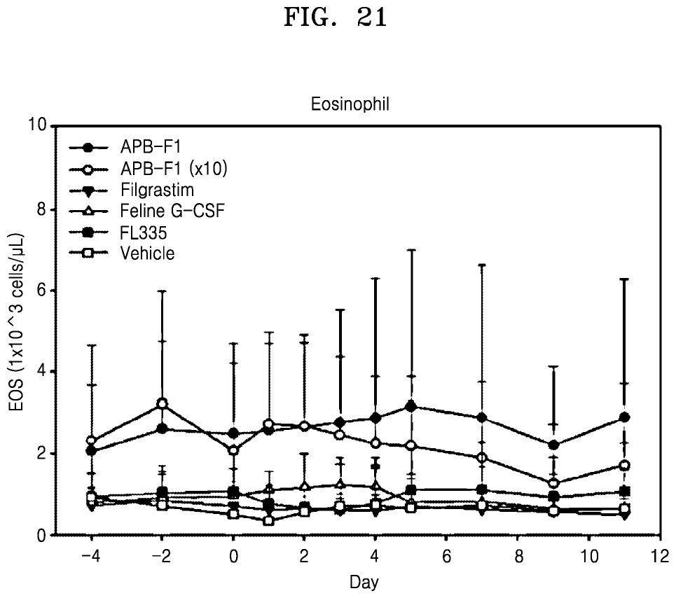

FIG. 20 shows results of examining lymphocyte levels in blood, and FIG. 21 shows results of examining eosinophil levels in blood. As shown in FIGS. 20 and 21, the lymphocyte and eosinophil levels showed no significant difference between experimental groups and control groups.

The results of this series of experiments indicate that APB-F1 according to one exemplary embodiment contributes to increasing the levels of white blood cells in blood, specifically, the levels of neutrophils, monocytes, and basophil in blood, and maintaining the levels for a long time.

A recombinant protein including an antigen binding fragment binding to serum albumin; and a feline granulocyte colony-stimulating factor can have improved pharmacokinetic properties including increased in-vivo half-life, and can continuously exhibit an effect of increasing white blood cell levels in blood to a therapeutically effective level.

Accordingly, the recombinant proteins disclosed herein can be used as active ingredients of compositions for preventing or treating feline panleukopenia.

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents. All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be herein incorporated by reference.

1. A recombinant protein comprising (a) an antigen binding fragment comprising a heavy chain and a light chain and (b) a feline granulocyte colony-stimulating factor (fGCSF),
wherein the heavy chain comprises a heavy chain variable domain and a feline heavy chain constant 1 domain, wherein the heavy chain variable domain comprises
(1) a heavy chain complementarity determining domain 1 (CDR1) comprising the amino acid sequence of SYGIS (SEQ ID NO:51),
a heavy chain complementarity determining domain 2 (CDR) comprising the amino acid sequence of WINTYSGGTKYAQKFQG (SEQ ID NO:52), and
a heavy chain complementarity determining domain 3 (CDR3) comprising the amino acid sequence of LGHCQRGICSDALDT (SEQ ID NO:53);
(2) a heavy chain CDR1 comprising the amino acid sequence of SYGIS (SEQ ID NO:51),
a heavy chain CDR2 comprising the amino acid sequence of RINTYNGNTGYAQRLQG (SEQ ID NO:54), and
a heavy chain CDR3 comprising the amino acid sequence of LGHCQRGICSDALDT (SEQ ID NO:53);
(3) a heavy chain CDR1 comprising the amino acid sequence of NYGIH (SEQ ID NO:55),
a heavy chain CDR2 comprising the amino acid sequence of SISYDGSNKYYADSVKG (SEQ ID NO:56), and
a heavy chain CDR3 comprising the amino acid sequence of DVHYYGSGSYYNAFDI (SEQ ID NO:57);
(4) a heavy chain CDR1 comprising the amino acid sequence of SYAMS (SEQ ID NO:58),
a heavy chain CDR2 comprising the amino acid sequence of VISHDGGFQYYADSVKG (SEQ ID NO:59), and
a heavy chain CDR3 comprising the amino acid sequence of AGWLRQYGMDV (SEQ ID NO:60);
(5) a heavy chain CDR1 comprising the amino acid sequence of AYWIA (SEQ ID NO:61),
a heavy chain CDR2 comprising the amino acid sequence of MIWPPDADARYSPSFQG (SEQ ID NO:62), and
a heavy chain CDR3 comprising the amino acid sequence of LYSGSYSP (SEQ ID NO:63); or
(6) a heavy chain CDR1 comprising the amino acid sequence of AYSMN (SEQ ID NO:64),
a heavy chain CDR2 comprising the amino acid sequence of SISSSGRYIHYADSVKG (SEQ ID NO:65), and
a heavy chain CDR3 comprising the amino acid sequence of ETVMAGKALDY (SEQ ID NO:66); and
wherein the light chain comprises a light chain variable domain and a feline light chain constant domain, wherein the light chain variable domain comprises
(7) a light chain CDR1 comprising the amino acid sequence of RASQSISRYLN (SEQ ID NO:67),
a light chain CDR2 comprising the amino acid sequence of GASRLES (SEQ ID NO:68), and
a light chain CDR3 comprising the amino acid sequence of QQSDSVPVT (SEQ ID NO:69);
(8) a light chain CDR1 comprising the amino acid sequence of RASQSISSYLN (SEQ ID NO:70),
a light chain CDR2 comprising the amino acid sequence of AASSLQS (SEQ ID NO:71), and
a light chain CDR3 comprising the amino acid sequence of QQSYSTPPYT (SEQ ID NO:72);
(9) a light chain CDR1 comprising the amino acid sequence of RASQSIFNYVA (SEQ ID NO:73),
a light chain CDR2 comprising the amino acid sequence of DASNRAT (SEQ ID NO:74), and
a light chain CDR3 comprising the amino acid sequence of QQRSKWPPTWT (SEQ ID NO:75);
(10) a light chain CDR1 comprising the amino acid sequence of RASETVSSRQLA (SEQ ID NO:76),
a light chain CDR2 comprising the amino acid sequence of GASSRAT (SEQ ID NO:77), and
a light chain CDR3 comprising the amino acid sequence of QQYGSSPRT (SEQ ID NO:78);
(11) a light chain CDR1 comprising the amino acid sequence of RASQSVSSSSLA (SEQ ID NO:79),
a light chain CDR2 comprising the amino acid sequence of GASSRAT (SEQ ID NO:77), and
a light chain CDR3 comprising the amino acid sequence of QKYSSYPLT (SEQ ID NO:80); or
(12) a light chain CDR1 comprising the amino acid sequence of RASQSVGSNLA (SEQ ID NO:81),
a light chain CDR2 comprising the amino acid sequence of GASTGAT (SEQ ID NO:82), and
a light chain CDR3 comprising the amino acid sequence of QQYYSFLAKT (SEQ ID NO:83).

2. The recombinant protein of claim 1, further comprising a linker that links the fGCSF to the antigen binding fragment.

3. The recombinant protein of claim 1, wherein (i) a cysteine in the feline heavy chain constant 1 domain and/or (ii) a cysteine in the feline light chain constant domain that is/are located in an interchain disulfide bond between the light chain and the heavy chain is/are conserved, deleted, and/or substituted with an amino acid residue other than cysteine.

4. The recombinant protein of claim 1,
wherein the heavy chain variable domain comprises a heavy chain CDR1 comprising the amino acid sequence of SEQ ID NO:64, a heavy chain CDR2 comprising the amino acid sequence of SEQ ID NO:65, and a heavy chain CDR3 comprising the amino acid sequence of SEQ ID NO:66, and
wherein the light chain variable domain comprises a light chain CDR1 comprising the amino acid sequence of SEQ ID NO:81, a light chain CDR2 comprising the amino acid sequence of SEQ ID NO:82, and a light chain CDR3 comprising the amino acid sequence of SEQ ID NO:83.

5. The recombinant protein of claim 1, wherein the heavy chain variable domain comprises an amino acid sequence having at least 80% identity to SEQ ID NO:1, 2, 3, 4, 5, or 6, and the light chain variable domain comprises an amino acid sequence having at least 80% identity to SEQ ID NO:7, 8, 9, 10, 11, 12, or 13.

6.-7. (canceled)

8. The recombinant protein of claim 1, wherein the feline heavy chain constant 1 domain comprises an amino acid sequence having at least 80% identity to SEQ ID NO:14, and the feline light chain constant domain comprises an amino acid sequence having at least 80% identity to SEQ ID NO:15.

9. (canceled)

10. The recombinant protein of claim 1, wherein the fGCSF is modified by removing a free cysteine group and an O-sugar chain from a naturally occurring fGCSF.

11. The recombinant protein of claim 1, wherein the fGCSF comprises an amino acid sequence having at least 80% identity to SEQ ID NO:18.

12. The recombinant protein of claim 1, wherein the fGCSF comprises an amino acid sequence having at least 80% identity to SEQ ID NO:19.

13. The recombinant protein of claim 1, wherein the fGCSF comprises the amino acid sequence of SEQ ID NO:19.

14. The recombinant protein of claim 2, wherein the linker links the fGCSF to a C-terminus of the feline heavy chain constant 1 domain, an N-terminus of the heavy chain variable domain, a C-terminus of the feline light chain constant domain, and/or an N-terminus of the light chain variable domain.

15. The recombinant protein of claim 2, wherein the linker comprises 1 to 50 amino acids.

16. A nucleic acid molecule encoding the recombinant protein of claim 1.

17. An expression vector comprising the nucleic acid molecule of claim 16.

18. A cell transformed with the expression vector of claim 17.

19. A composition comprising the recombinant protein of claim 1.

20. A pharmaceutical composition comprising the composition of claim 19 and a pharmaceutically acceptable excipient.

21. A kit comprising the composition of claim 19 and a label comprising instructions for a use.

22. A method of treating feline panleukopenia, comprising administering to a subject in need thereof the pharmaceutical composition of claim 20.

23. The method of claim 22, wherein the composition increases white blood cells in blood of the subject.

24.-25. (canceled)
